(19) United States
(12) Patent Application Publication
(10) Pub. No.: US 2017/0182221 A1
(43) Pub. Date: Jun. 29, 2017

(54) Methods Of Producing Tissue-Mimetic Constructs And Uses Thereof

(71) Applicants: National University of Singapore, Singapore (SG); Agency for Science, Technology and Research, Singapore (SG)

(72) Inventors: Paula-Beth Angelica Tiqui Benny, Singapore (SG); Michael Raghunath, Singapore (SG); Ellen Birgitte Lane, Singapore (SG); Cedric Badowski, Singapore (SG)

(21) Appl. No.: 15/308,713

(22) PCT Filed: May 5, 2015
(86) PCT No.: PCT/SG2015/050096

§ 371 Date: Nov. 3, 2016

Related U.S. Application Data
(60) Provisional application No. 61/988,709, filed on May 5, 2014.

(51) Int. Cl. A61L 27/52; A61L 27/36; A61L 27/38; C12N 5/071

(52) U.S. Cl. CPC A61L 27/52; C12N 5/0697; A61L 27/3633; A61L 27/3813; A61L 27/3804; C12N 2533/90

(57) ABSTRACT

The present invention relates, in various embodiments, to methods of producing a tissue-mimetic construct having a basement membrane, methods of producing an acellular scaffold containing an extracellular matrix (ECM), methods of producing a scaffold comprising a hydrogel that is enriched in ECM components, methods of treating a condition in a subject in need thereof with a tissue-mimetic construct having a basement membrane, and methods of assessing whether an agent is suitable for administering to a tissue. The invention further relates to tissue-mimetic constructs and scaffolds produced in accordance with the methods of the invention.

### RELATED APPLICATION

This application claims the benefit of U.S. Provisional Application No. 61/988,709, filed on May 5, 2014. The entire teachings of the above application are incorporated herein by reference.

### BACKGROUND OF THE INVENTION

The dermal-epidermal junction (DEJ) of the skin is a complex carpet of specialized extra cellular matrix (ECM) that provides anchorage for the waterproof epidermis to the mechanical buffering dermis below. Ultrastructurally, the basal keratinocyte layer of the epidermis is anchored to the type IV collagen-rich extracellular lamina densa via interactions with collagen XVII and laminin 332. The lamina densa is in turn fastened to the papillary dermis by anchoring fibrils, composed of collagen type VII that interlace with collagen I/III/V heterotypic fibrils of the dermis. This complex assembly of proteins is essential to maintain healthy skin tissue, and defects in any component results in tissue fragility disorders; for example, mutations in the collagen VII gene COL7A1 result in dystrophic epidermolysis bullosa.

Current tissue culture techniques for generating skin-mimetic structures suffer from the slow processing of collagen in vitro, and thus poor ECM formation. While fibronectin deposition is readily demonstrated in tissue culture, the deposition of collagen, the primary biological component essential for the basic structural formation of all tissues and organs, is enzymatically rate-limited such that it takes several weeks to produce tissue sheets which contain sufficient ECM in vitro. In addition, supramolecular assembly and deposition of collagen VII into a pericellular matrix has not been achieved experimentally in vitro.

Accordingly, there is a substantial need for the development of better tissue-mimetic constructs, including skin-mimetic constructs, which can be used for in vitro screening and toxicity assays, and to produce cultured autografts with improved efficacy for clinical applications.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-C: Mixed macromolecular crowding (mMMC) enhances deposition of dermal-epidermal junction components in vitro. FIG. 1A: Collagen I deposition is enhanced by mMMC (cell layer and matrix) in fibroblasts only. Crowding of co-cultures produce the most collagen I and show that keratinocytes stimulated collagen I production by fibroblasts. FIG. 1B: Collagen IV deposition by fibroblasts is enhanced by crowding. This is seen even more clearly in crowded co-cultures. Of note, keratinocytes stained for collagen IV show mostly cell-associated or intracellular collagen IV but not a pericellular matrix. In co-cultures, both cell types segregate with collagen IV being predominantly associated with fibroblasts sparing keratinocyte islands. FIG. 1C: Fibronectin deposition was only seen with fibroblasts, and therein strongly enhanced by crowding (cell layer and matrix). In co-cultures, a reticular mesh of fibronectin was associated with fibroblasts only, sparing islands of keratinocytes. Scale bars=20 μm.

FIG. 2: mMMC facilitates deposition of anchoring fibril building collagen VII. Panel A: A reticular deposition pattern of collagen VII deposition is evident with fibroblasts only under mMMC. In co-cultures, extracellular collagen VII is strongly associated with fibroblast colonies in between keratinocyte islands. Keratinocytes show pericellular and intracellular collagen VII more strongly expressed in the presence of mMMC. After cell lysis, collagen VII footprints are seen in a fine granular layer in mMMC treated fibroblast cultures, but a discernible fibrillar deposition is retrieved from co-cultures. B: Immunoblot analysis of lysed cell layers shows that both crowded fibroblasts and keratinocyte cultures contain significantly more collagen VII compared to uncrowded controls. The retrieved collagen VII is mainly pericellular-derived. C: Densitometric analysis of B shows that mMMC increases the amount of cell-associated collagen VII by a factor of 8 in fibroblasts and a factor of 2 in keratinocytes. Scale bars=20 μm.

FIG. 3: Keratinocytes adhere most effectively to f-Mat (generated under mMMC), produce the most amount of collagen VII and have the highest colony forming efficiency. Keratinocytes seeded on uncoated TCPS dishes produce little amounts of ECM (red=collagen VII). Decellularized uncrowded fibroblast cell layers, f-Mat, produce negligible amounts of ECM. Keratinocytes seeded on top of f-Mat produce significantly more ECM. B: Fibroblasts and keratinocytes were cultured with or without mMMC and subsequently decellularized to produce f-Mat and k-Mat respectively. Following that, keratinocytes were seeded on these matrices and observed for their colony forming efficiency. f-Mat (generated with mMMC) showed the highest capacity for keratinocyte colony formation.

FIG. 4: Fibroblast footprints contain more total ECM as visualized by IRM. On analysis of individual ECM components (collagen IV and fibronectin), culture under mMMC enhanced the extracellular deposition. To visualize the total ECM deposited, IRM was used to quantify all ECM as antibody stainings had their limitations. IRM clearly showed the total matrix quantity and pattern under mMMC as compared to control conditions.

FIG. 5: mMMC during the submerged phase enhances maturation of the DEJ in skin equivalents. Fibroblast-containing collagen gels where seeded with keratinocytes on top and kept submerged for one week, then lifted to air-liquid interface. In the classical protocol, collagen VII was absent after a total of 3 weeks in culture but appeared in skin equivalents after 5 weeks. In contrast, under mMMC, collagen VII was already strongly evident after 3 weeks and even more strongly stained after 5 weeks compared to standard cultures. H&E staining confirmed that with this rapid protocol, stratification and maturity of the skin equivalent were maintained and accelerated.

FIGS. 6A-C: Evidence of de novo formation of anchoring fibrils in skin equivalents generated under mMMC. Ultrastructural studies of the nascent dermal-epidermal junction of organotypic co-cultures after a 3 week culture protocol with mMMC (FIGS. 6A and 6B) suggests structures akin to anchoring fibrils (arrows) that are absent in non-crowded skin equivalents (FIG. 6C) after 3 weeks of culture.

FIG. 7: mMMC protocol allows development of a mature skin equivalent, with a proliferative and stratified epidermis. Keratin-10 expression is observed in skin equivalents cultured with macromolecular crowders for 3 weeks (red). Keratin-10 is also present in control skin equivalents after 3 weeks and 5 weeks. TGA is expressed in the organotypic co-cultures and appears most abundant in 3-week cultures with mMMC. Ki67 positive cells in the basal layer of organotypic co-cultures are most abundant in 3-week cultures. In human skin; TGA activity is localized to the basement membrane and upper epidermal layers of the skin; a double staining of Keratin-5 (basal layer) and Keratin-10 (suprabasal layer) differentiate the specific localization of these keratins in the skin; Ki67 (green) positive keratinocytes (nuclei=blue) in the basal layer of the epidermis of normal human skin. Scale bar=50 μm. The table below summarizes the list of markers to assess maturity of the skin equivalents. The “rapid” protocol (3 weeks) using Ficoll™ 70/400 produces a skin equivalent which has an enhanced extracellular matrix, marked by collagen VII, which results in anchoring fibril formation along the DEJ. Even though the culture time has been shortened from 5 weeks to 3 weeks, the maturity of the epidermis is still maintained as assessed by Keratin-10 (stratification) and transglutaminase activity. According to Ki67 assessment of the proliferative capacity of the basal keratinocytes, it is observed that the “rapid” protocol (3 weeks) with Ficoll™ 70/400 has more Ki67 positive cells as compared to uncrowded controls.

FIG. 8: Testing of potential agarose gel conjugate components for optimal cell growth (after 24 hours) and results obtained.

FIG. 9: Testing of potential agarose gel conjugate components for optimal cell growth (after 48 hours) and results obtained.

FIG. 10: Comparison of agarose and collagen gels as a scaffolding material for skin-mimetic structures.

FIG. 11: Methods of engineering an agarose-eECM gel and results obtained.

FIGS. 12A-C: Diagrams illustrating the production of an ECM that is concentrated on one surface (e.g., the top side) of a hydrogel (FIG. 12B), or is distributed evenly throughout the hydrogel (FIG. 12C). MMC=macromolecular crowders.

FIGS. 13A-C: Field emission scanning electron micrograph (FESEM) images of decellularized ECM derived from a fibroblast cell layer (f-Mat) that has been cultured with macromolecular crowders (MMCs) (FIG. 13A), a keratinocyte cell layer (k-Mat) that has been cultured with MMCs (FIG. 13B), or a co-culture of fibroblasts and keratinocytes (co-Mat) that have been cultured with MMCs (FIG. 13C).

FIG. 14: Atomic Force Microscopy (AFM) of co-Mat. Top image: Bright-field microscopy showing the AFM tip scanning over the cell-derived matrix (wispy network). Bottom image: AFM provided information on the mechanical properties of the cell-derived matrices. The Young's Modulus, or elastic modulus, measures stiffness and the co-Mat had an approximate Young's modulus of 1 kPa, falling under the category of “soft biomaterial”.

FIG. 15A: Images illustrating the surfaces of cell layers derived from cultures that were cultured without MMCs (left image) or with MMCs (right) prior to decellularization.

FIG. 15B: Image illustrating the thickness of the cell-derived matrix/Mats using a depth color-coding system.

FIGS. 16A and 16B: Colony forming assays of keratinocytes cultured on f-Mat (FIG. 16A) and co-Mat (FIG. 16B) matrices generated with macromolecular crowders.

FIGS. 17A and 17B: Pictures showing the handling of cell-derived matrices that were lacking (FIG. 17A) or had been mixed with (FIG. 17B) agarose.

FIGS. 18A-C: Merged IRM—immunofluorescent micrograph images showing the benefit of crowded-cell-derived matrices in terms of keratinocyte attachment. Keratinocytes are unable to adhere to an agarose hydrogel (FIG. 18A). Some keratinocytes were able to adhere to an agarose-uncrowded matrix hydrogel (FIG. 18B). Many more keratinocytes were able to adhere to the agarose-crowded matrix hydrogel (FIG. 18C). Blue: nuclei; Green: keratin.

FIG. 19: Picture showing pure cell-derived matrices that were collected, replated and incubated without (left) or with genipin (right).

FIG. 20: Graph showing that alginic acid and sodium alginate tended to form softer gels as compared to agarose and agarose-poly-lysine.

### DETAILED DESCRIPTION OF THE INVENTION

A description of example embodiments of the invention follows.

The present invention is based, in part, on the discovery that culturing epithelial cells and stromal cells together under macromolecular crowding conditions (e.g., in the presence of macromolecules of a particular size and concentration) can lead to the production of tissue-mimetic constructs. In particular aspects, the methods provided herein produce tissue-mimetic constructs that have a basement membrane.

Accordingly, the present invention relates, in one embodiment, to a method of producing a tissue-mimetic construct (e.g., a tissue-mimetic construct having a basement membrane). The method comprises the steps of: a) combining epithelial cells and stromal cells with one or more macromolecules having a hydrodynamic radius in the range of from about 2 nm to about 50 nm, thereby producing a mixed cell culture; and b) maintaining the cell culture under conditions in which the epithelial cells and stromal cells proliferate and produce a basement membrane, thereby producing a tissue-mimetic construct having a basement membrane.

As used herein, “tissue-mimetic construct” refers to a multicellular composition that comprises living cells attached to an extracellular matrix, and performs one or more functions of a living tissue (e.g., a selectively permeable barrier function, absorption of water and nutrients, elimination of waste product, specialized physiological functions, secretion of substances). A tissue-mimetic construct can be two-dimensional or three-dimensional. Exemplary tissue-mimetic constructs that can be produced by the methods described herein include, but are not limited to, a skin-mimetic construct, a corneal tissue-mimetic construct, an oral mucosa-mimetic construct, a bladder tissue-mimetic construct, a vaginal tissue-mimetic construct, an esophageal tissue-mimetic construct, a liver tissue-mimetic construct, a pancreatic tissue-mimetic construct, a kidney tissue-mimetic construct, a lung tissue-mimetic construct, a gingival tissue-mimetic construct and a cartilage tissue-mimetic construct.

One of ordinary skill in this art can readily identify suitable epithelial cells and stromal cells for producing a tissue-mimetic construct for a particular tissue type. For example, to produce a corneal tissue-mimetic construct with a basement membrane according to a method of the invention, corneal epithelial cells and corneal stromal cells that make basement membranes can be selected and combined in a mixed cell culture. As used herein, the terms “mixed cell culture” and “co-culture” are used interchangeably to refer to a cell culture containing two or more cell types (e.g., epithelial cells and stromal cells).

In certain aspects, the methods of producing a tissue-mimetic construct (e.g., a tissue-mimetic construct having a basement membrane) include combining epithelial and stromal cells to produce a mixed cell culture. “Epithelial cells” are cells that are bound together in sheets of epithelia that line the major cavities of the body. “Stromal cells” are cells found in connective tissue.

In particular aspects, a mixed cell culture is produced by seeding one cell type (a first cell type) over a different cell type (a second cell type). In one embodiment, a mixed cell culture is produced by seeding epithelial cells (e.g., keratinocytes) over stromal cells (e.g., fibroblasts). In another embodiment, a mixed cell culture is produced by seeding stromal cells (e.g., fibroblasts) over epithelial cells (e.g., keratinocytes). In yet another embodiment, a mixed cell culture is produced by seeding epithelial and stromal cells together at the same time.

In a particular aspect, the tissue-mimetic construct is a skin-mimetic construct. In one embodiment, the skin-mimetic construct is an organotypic skin culture. “Organotypic culture” refers to a cell culture in which cells are grown in a three-dimensional environment. In another embodiment, the skin-mimetic construct is a cultured skin graft (e.g., a cultured autograft, cultured allograft, a cultured xenograft). As used herein, the terms “cultured skin graft,” “cultured epithelial autograft” (CEA), and “living skin equivalent” (LSE) are used interchangeably to refer to a skin graft that is produced using cultured cells.

In one aspect, a mixed cell culture comprising epithelial skin cells and stromal skin cells is used in a method of producing a skin-mimetic construct. Epithelial skin cells include, for example, keratinoctyes. The keratinocytes can be epidermal keratinocytes (e.g., keratinocytes isolated from skin, hair, or nails), keratinocytes derived from induced pluripotent cells (iPS), or keratinocytes derived from human embryonic stem cells (hESCs), or a combination thereof. In a particular aspect, the keratinocytes are human keratinocytes (e.g., human primary keratinocytes).

Exemplary stromal skin cells for preparing a skin-mimetic construct include, among others, fibroblasts. The fibroblasts can be, for example, dermal fibroblasts, fibroblasts derived from induced pluripotent stem cells (iPS), or fibroblasts derived from human embryonic stem cells (hESCs), or any combination thereof. In a particular aspect, the fibroblasts are human fibroblasts (e.g., human primary fibroblasts).

Other suitable skin cells for producing a skin-mimetic construct (e.g., a skin-mimetic construct with a basement membrane) include, but are not limited to, epithelial stem cells, melanocytes, microvascular endothelial cells, dermal mesenchymal stem cells (DMSCs) (e.g., DMSCs derived from bone marrow), dermal pericytes, and dermal cells derived from adipose tissue.

In accordance with the invention, the method of producing a tissue-mimetic construct having a basement membrane comprises combining the epithelial cells and stromal cells with one or more macromolecules. Typically, macromolecules that are suitable for use in the methods described herein have a hydrodynamic radius in the range of about 2 nm to about 50 nm Such macromolecules can have a molecular weight in the range of about 50 kDa to about 1000 kDa.

Suitable macromolecules for use in the methods of the invention include, for example, carbohydrates, proteins, graphene, and synthetic polymers (e.g., polyvinylpyrrolidone, polyethylene glycol). Such macromolecules can be naturally occurring, recombinant or synthetic. In a particular aspect, the concentration of each of the one or more macromolecules in the mixed cell culture is from about 2.5 mg/ml to about 100 mg/ml.

In a particular embodiment, the one or more macromolecules are carbohydrate-based macromolecules. As used herein, a “carbohydrate-based macromolecule” refers to an inert molecule that contains at least one carbohydrate component and has a hydrodynamic radius in the range of from about 2 nm to about 50 nm. In a particular aspect, the one or more carbohydrate-based macromolecules include at least one polymer of glucose, at least one polymer of sucrose (e.g., at least one Ficoll sucrose polymer), or a combination thereof. In a further aspect, the one or more carbohydrate-based macromolecules includes two or more sucrose polymers of different molecular weights comprising the following structural components:

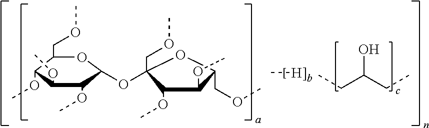

wherein n is an integer greater than 1.

Non-limiting examples of carbohydrate-based macromolecules include Ficoll™ PM70, Ficoll™ PM400, polyvinyl pyrrolidone, polyethylene glycol (PEG), dextran, dextran sulfate, polystyrene sulfonate, pullulan, and fucoidan, as well as combinations thereof. In a particular embodiment, the one or more carbohydrate-based macromolecules include a combination of Ficoll™ PM70 and Ficoll™ PM400.

The one or more macromolecules can be added to a culture of epithelial cells or stromal cells, or to a mixed cell culture of epithelial and stromal cells. In one embodiment, a mixed cell culture is produced by adding epithelial cells (e.g., keratinocytes) to a medium that contains stromal cells (e.g., fibroblasts) and one or more macromolecules. In another embodiment, a mixed cell culture is produced by adding stromal cells (e.g., fibroblasts) to a medium that contains epithelial cells (e.g., keratinocytes) and one or more macromolecules. In yet another embodiment, a mixed cell culture is produced by combining epithelial and stromal cells and subsequently adding one or more carbohydrate-based macromolecules.

In some embodiments, the mixed cell culture further includes a scaffold (e.g., for cell seeding and attachment). In a particular aspect, the scaffold is a hydrogel. Particular hydrogels that are suitable for use as scaffolds in the methods described herein include, for example, collagen gels, fibrin gels, agarose gels, hyaluronic acid gels, polyethylene glycol gels, alginate gels (e.g., alginic acid, sodium alginate) and cellulose gels (e.g., bacterial cellulose).

In one embodiment, the scaffold includes a structurally reinforced hydrogel. Suitable means and materials for reinforcing a hydrogel are well known in the art. In various aspects, the hydrogel can be reinforced, for example, using a synthetic reinforcement (e.g., a nanofiber) or additional scaffolding, or by crosslinking the molecules in the hydrogel with chemicals or UV irradiation.

In certain aspects, the mixed cell culture containing the one or more carbohydrate-based macromolecules is interspersed throughout the scaffold (e.g., hydrogel). In other aspects, the mixed cell culture containing the one or more carbohydrate-based macromolecules is applied to one side (e.g., the top side) of the scaffold.

In accordance with the invention, the method of producing a tissue-mimetic construct having a basement membrane further comprises maintaining the cell culture under conditions in which the epithelial cells and stromal cells proliferate and produce a basement membrane.

As used herein, the term “basement membrane” refers to a layer of extracellular matrix (ECM) that underlies a tissue epithelium and is produced by the concerted action of epithelial cells and stromal cells. Typically, the ECM in a basement membrane will include ECM components such as, for example, laminins, fibronectins, elastins, collagens (types I-XIII), and proteoglycans (e.g., heparin sulfate proteoglycans). The ECM composition in a basement membrane can vary according to tissue type. For example, a skin-mimetic construct will typically be enriched in one or more extracellular matrix components selected from the group consisting of collagen I, collagen IV, collagen VII, fibronectin and laminin 332/laminin 5, or a combination thereof.

Conditions in which the epithelial cells and stromal cells proliferate and produce a basement membrane are known in the art and include, for example, conditions described in International Publication Number WO 2011/108993 A1, the contents of which are incorporated herein by reference. Other exemplary conditions are described in Example 1 herein. In a particular aspect, the epithelial cells and stromal cells are cultured in the presence of ascorbic acid (e.g., 10004 ascorbic acid), which can be added to the culture medium.

In one embodiment, the conditions under which the epithelial cells and stromal cells proliferate and produce a basement membrane include submerging the cell culture in a medium containing one or more macromolecules (e.g., carbohydrate-based macromolecules), thereby producing a submerged culture. As used herein, a “submerged culture” is a cell culture in which the cells are immersed in a liquid medium. In a particular aspect, the submerged culture is maintained for a period of about 2 days to about 14 days, preferably about 7 days.

Such conditions can further comprise raising the submerged culture to the gas-liquid interface, thereby producing a raised culture. “Raised culture” refers to a cell culture that is grown at a gas (e.g., air, oxygen)-liquid interface. In a particular aspect, the raised culture is maintained at the gas-liquid interface for about one week, about two weeks, about three weeks, about four weeks or about five weeks. In another particular aspect, the raised culture is maintained at the gas-liquid interface for about two weeks.

In one embodiment, the tissue-mimetic construct is a skin-mimetic construct and the method comprises maintaining the cell culture under conditions in which keratinocytes and fibroblasts proliferate and produce a basement membrane that is enriched in one or more extracellular matrix components selected from the group consisting of collagen I, collagen IV, collagen VII, fibronectin and laminin 332/laminin 5, or a combination thereof. In a particular aspect, the basement membrane in the skin-mimetic construct is enriched in collagen VII. In a particular embodiment, the conditions under which the keratinocytes and fibroblasts proliferate and produce a basement membrane are sufficient to produce a skin-mimetic construct with a stratified epidermis, anchoring fibrils or a combination thereof. “Stratified epidermis” refers to an epidermis having vertically stacked layers of epithelial cells that have different functions and protein expression profiles depending on their position in the stack. In another embodiment, the conditions under which the keratinocytes and fibroblasts proliferate and produce a basement membrane are sufficient to produce a skin-mimetic construct in a time period of about three weeks.

### Tissue-Mimetic Constructs and Uses Thereof

In other embodiments, the invention encompasses a tissue-mimetic construct having a basement membrane, wherein the tissue-mimetic construct is made by a method described herein. In a preferred embodiment, the tissue-mimetic construct is a skin-mimetic construct (e.g., a skin-mimetic construct having anchoring fibrils, a skin-mimetic construct having a stratified epidermis). Such tissue-mimetic constructs are useful for a variety of purposes including, but not limited to, assays, methods of assessing whether an agent is suitable for administering to a tissue and methods of treating a condition affecting or involving a tissue in a subject.

Thus, the invention also provides methods of using a tissue-mimetic construct produced by the methods described herein. In a particular embodiment, the invention relates to a method of performing an assay on a tissue-mimetic construct produced by a method of described herein. Such assays can be an in vitro assay, an ex vivo assay or an in vivo assay. Assays that can be performed on a tissue-mimetic construct described herein include, but are not limited to, drug screening assays, toxicity testing assays, disease modeling, wound healing assays, and tissue grafting assays.

In another embodiment, the invention relates to a method of assessing whether an agent is suitable for administering to a tissue (e.g., skin), comprising the steps of contacting a tissue-mimetic construct produced according to a method described herein with an agent to be assessed and determining whether the agent produces a desired effect (e.g., no effect, an improved effect) on the tissue-mimetic construct compared to a control. In accordance with the invention, if the agent produces a desired effect on the tissue-mimetic construct compared to a control, then the agent is suitable for administering to the tissue. As will be apparent to one of ordinary skill in the art, a variety of suitable controls can be used. For example, a suitable control includes a tissue-mimetic construct that has not been contacted with the agent to be assessed.

The agent to be tested can be an organic, inorganic or organometallic compound (e.g., a small molecule), or a biological macromolecule, among others. Such agents can be naturally occurring, recombinant or synthetic.

Desired effects include, for example, a therapeutic effect (e.g., a wound healing effect, a tissue regeneration effect) and a lack of an undesired effect (e.g., lack of toxicity, absence of harmful side effects, such as a lack of irritation or inflammation).

Tissue-mimetic constructs produced by the methods described herein also can be therapeutically applied to a living subject to treat various conditions (e.g., conditions involving a tissue pathology). Thus, in certain embodiments, the invention relates to a method of applying a tissue-mimetic construct produced according to a method described herein to a subject in need thereof. As used herein, “subject” refers to a mammalian subject (e.g., human, non-human primate, horse, camel, cow, pig, dog, cat, mouse, rat). In a particular aspect, the subject is a human. A “subject in need thereof” refers to a subject who has, or is at risk for developing, a tissue pathology that can be treated by application of a tissue-mimetic construct made by a method described herein. For example, a subject in need thereof can be a subject with a wound or burn that can be treated by applying a skin-mimetic construct having a basement membrane.

In a related embodiment, the invention relates to a method of treating a condition (e.g., a condition involving a tissue pathology) in a subject in need thereof, comprising applying a tissue-mimetic construct produced by a method of the invention to a subject in need thereof. In one embodiment, the condition to be treated is a tissue lesion (e.g., a skin lesion, a corneal lesion, an oral lesion, a bladder lesion, a vaginal lesion, an esophageal lesion, a liver lesion, a pancreatic lesion, a kidney lesion, a lung lesion) and a tissue-mimetic construct of the corresponding tissue-type (e.g., skin, corneal, oral mucosal, bladder, vaginal, esophageal, liver, pancreas, kidney, lung) is applied to the subject. In one embodiment, the tissue lesion is an ulcer.

In a particular embodiment, the condition to be treated is a skin condition and a skin-mimetic construct is applied to the subject. Such skin-mimetic constructs can function as, for example, a skin graft or wound dressing. Skin conditions to be treated include, but are not limited to, wounds and burns. The wound to be treated can be an open wound or a closed wound and, furthermore, can be acute or chronic and, additionally, can be healing or non-healing. Exemplary wounds to be treated include abrasions, blisters, bruises, and puncture wounds (e.g., bite wounds, stab wounds)

### Methods of Producing Scaffolds Enriched in Extracellular Matrix Components

The invention further provides, in another embodiment, a method of producing an acellular scaffold containing an extracellular matrix. As used herein “acellular scaffold” refers to a non-living structure that comprises an extracellular matrix and which functions as a substrate for the attachment of living cells. Such acellular scaffolds are useful as platforms for secondary cell (e.g., stem cell) seeding and propagation, as a microenvironment for cell attachment and growth, and as means for studying cell function and conducting assays (e.g., toxicity assays). In a particular aspect, the acellular scaffold is seeded with stem cells, including, but not limited to, embryonic stem cells, induced pluripotent stem cells, epithelial stem cells, epidermal stem cells. In a particular embodiment, the stem cells are human stem cells.

An acellular scaffold produced by a method described herein is enriched in one or more ECM components such as, but not limited to, laminins, fibronectins, elastins, collagens (e.g., collagen types I-XIII), and proteoglycans (e.g., heparin sulfate proteoglycans). A skin-mimetic construct will typically be enriched in one or more extracellular matrix components selected from the group consisting of collagen I, collagen IV, collagen VII, fibronectin and laminin 332/laminin 5, or a combination thereof. In a particular embodiment, the acellular scaffold further comprises a hydrogel.

In accordance with the invention, the method of producing an acellular scaffold comprises the steps of a) combining epithelial cells, stromal cells and one or more macromolecules, thereby producing a mixed cell culture; b) maintaining the cell culture under conditions in which the epithelial cells and stromal cells proliferate and produce an extracellular matrix; and c) decellularizing the cell culture.

In one embodiment, the mixed cell culture includes skin epithelial cells (e.g., keratinocytes) and stromal cells (e.g., fibroblasts). In another embodiment, the mixed cell culture includes a combination of epithelial cells and stromal cells selected from the group consisting of a combination of corneal epithelial cells and stromal cells, a combination of oral mucosal epithelial cells and stromal cells, a combination of liver epithelial cells and stromal cells, a combination of pancreatic epithelial cells and stromal cells, a combination of kidney epithelial cells and stromal cells, a combination of bladder epithelial cells and stromal cells, and a combination of lung epithelial cells and stromal cells.

Techniques and reagents for decellularizing a cell culture are well known and include, for example, cell lysis techniques and reagents. Suitable cell lysis techniques include, for example, subjecting the cells to freeze-thaw cycling, treating the cells with one or more cell lysis agents, homogenizing the cells and sonicating the cells. Preferred cell lysis reagents include detergents (e.g., sodium deoxycholate, nonyl phenoxypolyethoxylethanol (NP-40), octylphenoxypolyethoxyethanol), nucleases (DNases, RNases), salts (e.g., NaCl, KCl) and ammonium hydroxide, among others. Exemplary cell lysis techniques are described in Lu, H., et al., J. Biomed. Mater. Res. A 100(9):2507-2516 (2012), the contents of which are incorporated herein by reference.

In an additional embodiment, the invention relates to a method of producing a scaffold comprising a hydrogel that is enriched in extracellular matrix (ECM) components. In accordance with the invention, the method comprises preparing an enriched extracellular matrix by culturing epithelial cells, stromal cells, or a combination thereof with one or more macromolecules having a hydrodynamic radius in the range of from about 2 nm to about 50 nm; and combining the enriched extracellular matrix with a hydrogel, thereby producing a scaffold comprising a hydrogel that is enriched in ECM components. In a further embodiment, the method can additionally comprise the step of decellularizing the hydrogel scaffold after it has been enriched in ECM, e.g., using a method or technique described herein (e.g., cell lysis).

Particular hydrogels that are suitable for use as scaffolds enriched in ECM components include, for example, collagen gels, fibrin gels, agarose gels, hyaluronic acid gels, polyethylene glycol gels, alginate gels (e.g., alginig acid, sodium alginate) and cellulose gels (e.g., bacterial cellulose gels).

In one embodiment, the hydrogel comprises agarose or an agarose conjugate. In a particular aspect, the hydrogel contains a percentage of agarose in the range of about 0.1% to about 20%, preferably in the range of about 0.5% to about 5% agarose, and more preferably in the range of about 1.25% to about 2.5% agarose.

In a particular embodiment, the scaffold comprises one or more hydrogel conjugates. “Hydrogel conjugate” refers to a hydrogel polymer that is covalently attached to one or more non-hydrogel molecules (e.g., glycoproteins, amino acids, growth factors). In a preferred embodiment, the scaffold comprises a hydrogel containing one or more agarose conjugates (e.g., agarose-lysine conjugates, agarose-fibronectin conjugates, agarose-lysine-fibronectin conjugates, or a combination thereof).

In a particular aspect, the hydrogel scaffold is enriched in one or more extracellular matrix components selected from the group consisting of collagen I, collagen IV, collagen VII, fibronectin and laminin 332/laminin 5, or a combination thereof. In a particular embodiment, the scaffold is enriched in collagen VII.

Preferred epithelial cells are keratinocytes. Preferred stromal cells are fibroblasts (e.g., dermal fibroblasts).

In a particular embodiment, the method comprises preparing an enriched extracellular matrix by culturing stromal cells (e.g., fibroblasts) with one or more macromolecules (e.g., carbohydrate-based macromolecules) having a hydrodynamic radius in the range of from about 2 nm to about 50 nm, combining the enriched extracellular matrix with a hydrogel and subsequently seeding and maintaining epithelial cells (e.g., keratinoctyes) on the enriched ECM scaffold under conditions in which the epithelial cells attach to the hydrogel and spread.

In certain aspects, the ECM is interspersed throughout the enriched ECM scaffold (e.g., hydrogel). For example, ECM (e.g., ECM produced by culturing epithelial cells, stromal cells, or a combination thereof with one or more macromolecules having a hydrodynamic radius in the range of from about 2 nm to about 50 nm) can be mixed with the scaffold material (e.g., hydrogel), thereby ensuring an approximately even distribution of the ECM throughout the scaffold.

In other aspects, the ECM is localized to a portion of the enriched ECM scaffold. Without wishing to be bound by any one theory, it is believed that the use of a scaffold having an enriched ECM that is localized to a portion (e.g., side) of the scaffold can support superior cell attachment and migration relative to scaffolds interspersed with ECM.

Thus, in a particular aspect, the ECM is concentrated on (e.g., stamped on, applied to) one side (e.g., the top side) of a scaffold. For example, epithelial cells, stromal cells, or a combination thereof can be cultured with one or more macromolecules having a hydrodynamic radius in the range of about 2 nm to about 50 nm on a thermosensitive surface (e.g., a surface comprising thermosensitive molecules, such as thermosensitive polymers). The ECM produced by the cell culture can then be attached to the scaffold and harvested (e.g., isolated) by altering (e.g., lowering) the temperature of the thermosensitive surface, thereby detaching the ECM intact (e.g., as a sheet) from the thermosensitive surface (e.g., without the need for scraping). Suitable thermosensitive cell culture surfaces for isolating intact ECM include, but are not limited to, Nunc™ Dishes with UpCell™ Surface (Thermo Fisher Scientific, Inc., Waltham, Mass.).

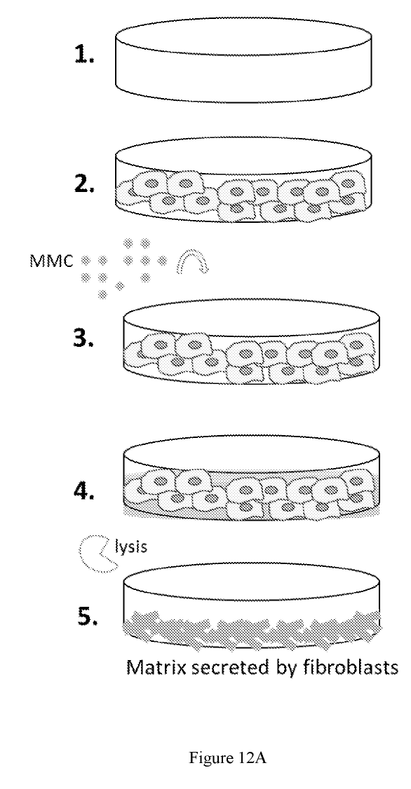

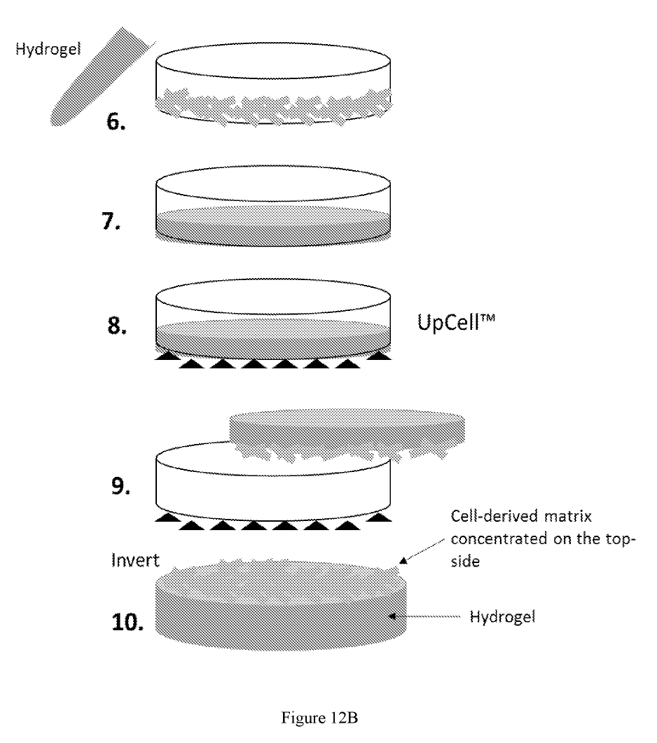

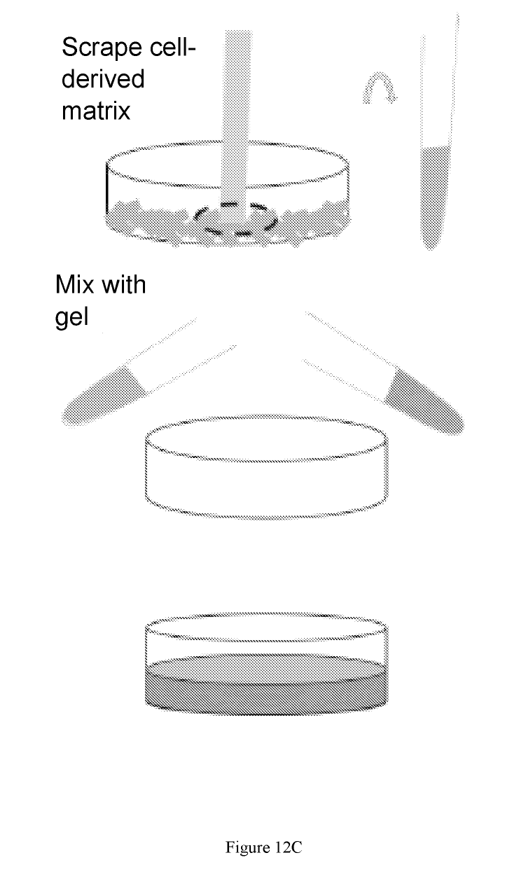

In one aspect, the scaffold (e.g., hydrogel) is applied to the top of an ECM that is still attached to the thermosensitive surface (e.g., after the cell culture has been removed, for example, by cell lysis). Once the scaffold has been applied, the ECM with the scaffold attached can be released from the thermosensitive surface, thereby producing an ECM that is concentrated on one side of the scaffold (e.g., the top side of a hydrogel) (FIG. 12). In another aspect, the intact ECM is detached from the thermosensitive surface before the scaffold is attached.

Thus, in another embodiment, the invention relates to a method of producing a scaffold (e.g., hydrogel) that is enriched in extracellular matrix (ECM) components, wherein the ECM components are concentrated on the surface of the scaffold. According to the invention, the method comprises a) preparing an enriched extracellular matrix by culturing epithelial cells, stromal cells, or a combination thereof with one or more macromolecules having a hydrodynamic radius in the range of from about 2 nm to about 50 nm and b) attaching the enriched extracellular matrix to the surface of the scaffold, thereby producing a scaffold that is enriched in ECM components on the surface. In a particular embodiment, the scaffold is a hydrogel.

In one aspect, the epithelial cells, stromal cells, or a combination thereof are cultured with the one or more macromolecules on a thermosensitive surface, and the enriched ECM is released from the thermosensitive surface by altering the temperature of the thermosensitive surface. In one embodiment, the enriched ECM is attached to the scaffold before it is released from the thermosensitive surface. In another embodiment, the enriched ECM is released from the thermosensitive surface before it is attached to the scaffold.

In other aspects of the invention, the invention relates to scaffolds (e.g., acellular scaffolds, hydrogel scaffolds enriched in ECM components) produced by the methods described herein.

### Additional Embodiments of the Invention

In one embodiment, the invention relates to a method of producing a tissue-mimetic construct having a basement membrane, comprising:
a) combining epithelial cells and stromal cells with one or more macromolecules having a hydrodynamic radius in the range of from about 2 nm to about 50 nm, thereby producing a mixed cell culture; and
b) maintaining the cell culture under conditions in which the epithelial cells and stromal cells proliferate and produce a basement membrane,
thereby producing a tissue-mimetic construct having a basement membrane.

In an aspect of this embodiment, the tissue-mimetic construct is a skin-mimetic construct, a corneal tissue-mimetic construct, an oral mucosa-mimetic construct, a bladder tissue-mimetic construct, a liver tissue-mimetic construct, a pancreatic tissue-mimetic construct, a kidney tissue-mimetic construct, or a lung tissue-mimetic construct.

In another aspect of this embodiment, the tissue-mimetic construct is a skin-mimetic construct, the epithelial cells are keratinocytes and the stromal cells are fibroblasts.

In a further aspect of this embodiment, the basement membrane is enriched in at least one extracellular matrix component selected from the group consisting of collagen I, collagen IV, collagen VII, fibronectin and laminin 332/laminin 5, or a combination thereof. In a particular aspect, the basement membrane is enriched in collagen VII.

In an additional aspect of this embodiment, the concentration of each of the one or more macromolecules is from about 2.5 mg/ml to about 100 mg/ml.

In yet another aspect of this embodiment, each of the one or more macromolecules has a molecular weight from about 50 kDa to about 1000 kDa.

In certain aspects of this embodiment, the one or more macromolecules are carbohydrate-based macromolecules. In a particular aspect, the one or more carbohydrate-based macromolecules includes at least one polymer of glucose, at least one polymer of sucrose, or a combination thereof. In a further aspect, the one or more carbohydrate-based macromolecules includes Ficoll™ PM70, Ficoll™ PM400, polyvinyl pyrrolidone, dextran, dextran sulfate, polystyrene sulfonate, pullulan, or fucoidan, or a combination thereof. In one aspect, the one or more carbohydrate-based macromolecules includes a combination of Ficoll™ PM70 and Ficoll™ PM400.

In various aspects of this embodiment, the fibroblasts are dermal fibroblasts, fibroblasts derived from induced pluripotent cells (iPS), or fibroblasts derived from human embryonic stem cells (hESCs), or a combination thereof.

In some aspects of this embodiment, the keratinocytes are epidermal keratinocytes, keratinocytes derived from induced pluripotent cells (iPS), or keratinocytes derived from human embryonic stem cells (hESCs), or a combination thereof.

In a particular aspect of this embodiment, the fibroblasts are human fibroblasts and the keratinocytes are human keratinocytes. In further aspect, the human fibroblasts are human primary fibroblasts and the human keratinocytes are human primary keratinocytes.

In one aspect of this embodiment, the keratinoctyes are seeded over the fibroblasts. In another aspect of this embodiment, the fibroblasts are seeded over the keratinocytes.

In yet another aspect of this embodiment, the mixed cell culture includes a scaffold. In one aspect, the scaffold is a hydrogel. In certain aspects, the hydrogel comprises collagen, fibrin, agarose, hyaluronic acid, polyethylene glycol, alginate or cellulose.

In a particular aspect of this embodiment, the conditions in which the keratinocytes and fibroblasts proliferate and produce a basement membrane are suitable for forming anchoring fibrils.

In some aspects of this embodiment, the keratinocytes and fibroblasts are submerged in a medium containing the one or more carbohydrate-based macromolecules, thereby producing a submerged culture. In a particular aspect, the submerged culture is maintained for about one week.

In a particular aspect of this embodiment, the method further comprises raising the submerged culture to the air-liquid interface, thereby producing a raised culture. In one aspect, the raised culture is maintained at the air-liquid interface for about one week, about two weeks or about three weeks.

In one aspect of this embodiment, a skin-mimetic construct having a basement membrane is produced in about three weeks.

In another aspect of this embodiment, a skin-mimetic construct having a stratified epidermis is produced by the method.

In a further aspect of this embodiment, a skin-mimetic construct containing anchoring fibrils is produced.

In various aspects of this embodiment, the skin-mimetic construct is an organotypic skin culture or a cultured skin graft. In a particular aspect, the cultured skin graft is a cultured autograft. In another aspect, the cultured skin graft is a cultured allograft. In yet another aspect, the cultured skin graft is a cultured xenograft.

In one aspect of this embodiment, the keratinocytes are added to a medium that contains the fibroblasts and the one or more macromolecules.

In another aspect of this embodiment, the fibroblasts are added to a medium that contains the keratinocytes and the one or more macromolecules.

In yet another aspect of this embodiment, the keratinocytes and fibroblasts are mixed with each other prior to being combined with the one or more carbohydrate-based macromolecules. In some aspects of this embodiment, the method further comprises applying the tissue-mimetic construct to a subject in need thereof. In a particular aspect, the subject is a human. In another aspect, the subject is a non-human mammal.

In certain aspects of this embodiment, the method further comprises performing an in vitro screening assay or toxicity assay on the tissue-mimetic construct.

In an additional embodiment, the invention relates to tissue-mimetic construct produced by a method of the preceding embodiment.

In another embodiment, the invention relates to a method of producing an acellular scaffold containing an extracellular matrix, comprising:
a) combining epithelial cells, stromal cells and one or more carbohydrate-based macromolecules, thereby producing a mixed cell culture;
b) maintaining the cell culture under conditions in which the epithelial cells and stromal cells proliferate and produce an extracellular matrix; and
c) decellularizing the cell culture,
thereby producing an acellular scaffold containing an extracellular matrix.

In an aspect of this embodiment, the epithelial cells are keratinocytes.

In an additional aspect of this embodiment, the stromal cells are fibroblasts.

In one aspect of this embodiment, the cell culture is decellularized by lysing the cells in the culture. In a further aspect, lysing the cells in the culture comprises subjecting the cells to freeze-thaw cycling, treating the cells with one or more agents selected from the group consisting of a detergent, a deoxyribonuclease, a salt, and ammonium hydroxide, or a combination thereof. In a particular aspect, the detergent is sodium deoxycholate, nonyl phenoxypolyethoxylethanol (NP-40), or octylphenoxypolyethoxyethanol and the salt is NaCl or KCl.

In another aspect of this embodiment, the extracellular matrix includes at least one component selected from the group consisting of collagen I, collagen IV, collagen VII, fibronectin and laminin 332/laminin 5, or a combination thereof.

In another embodiment, the invention relates to an acellular scaffold produced by a method of the preceding embodiment.

In an additional embodiment, the invention provides a method of producing a scaffold comprising a hydrogel that is enriched in extracellular matrix (ECM) components, comprising:
a) preparing an enriched extracellular matrix by culturing epithelial cells, stromal cells, or a combination thereof with one or more macromolecules having a hydrodynamic radius in the range of from about 2 nm to about 50 nm; and
b) combining the enriched extracellular matrix with a hydrogel, thereby producing a scaffold comprising a hydrogel that is enriched in ECM components.

In an aspect of this embodiment, the hydrogel is a collagen gel, a fibrin gel, an agarose gel, a hyaluronic acid gel, a polyethylene glycol gel, an alginate gel or a cellulose gel. In a particular aspect, the hydrogel is an agarose gel. In a further aspect, the agarose gel contains a percentage of agarose in the range of about 0.5% to about 20%. In one aspect, the agarose gel contains about 1.25% agarose to about 2.5% agarose.

In some aspects of this embodiment, the agarose gel comprises an agarose conjugate. In certain aspects, the agarose conjugate is an agarose-lysine conjugate, an agarose-fibronectin conjugate, an agarose-lysine-fibronectin conjugate, or a combination thereof.

In a particular aspect of this embodiment, the epithelial cells are keratinocytes.

In a certain aspect of this embodiment, the stromal cells are fibroblasts.

In some aspects of this embodiment, the method further comprises seeding keratinoctyes on the scaffold and maintaining the keratinocytes under conditions in which the keratinocytes attach and spread.

In another embodiment, the invention relates to a scaffold produced by a method of the preceding embodiment.

In an additional embodiment, the invention relates to a method of treating a condition in a subject in need thereof, comprising producing a tissue-mimetic construct according to the method of any one of claims 1-39 and applying the tissue-mimetic construct to the skin of the subject, thereby treating the condition.

In an aspect of this embodiment, the condition is a tissue lesion.

In another aspect of this embodiment, the condition is a skin wound.

In some aspects of this embodiment, the subject is a human.

In other aspects of this embodiment, the subject is a non-human mammal.

In a further embodiment, the invention relates to a method of assessing whether an agent is suitable for administering to a tissue, comprising:
a) contacting a tissue-mimetic construct produced according to the method of any one of claims 1-39 with an agent to be assessed;
b) determining whether the agent produces a desired effect on the tissue-mimetic construct compared to a control,
wherein if the agent produces a desired effect on the tissue-mimetic construct compared to a control, then the agent is suitable for administering to the tissue.

In an aspect of this embodiment, the tissue-mimetic construct is a skin-mimetic construct.

In one aspect of this embodiment, the desired effect is lack of toxicity.

In another aspect of this embodiment, the desired effect is a wound healing effect.

In yet another embodiment, the invention relates to a method of producing a scaffold that is enriched in extracellular matrix (ECM) components, wherein the ECM components are concentrated on the surface of the scaffold, comprising:
a) preparing an enriched extracellular matrix by culturing epithelial cells, stromal cells, or a combination thereof with one or more macromolecules having a hydrodynamic radius in the range of from about 2 nm to about 50 nm; and
b) attaching the enriched extracellular matrix to the surface of the scaffold,
thereby producing a scaffold that is enriched in ECM components on the surface.

In an aspect of this embodiment, the epithelial cells, stromal cells, or combination thereof are cultured with the one or more macromolecules on a thermosensitive surface, and the enriched ECM is isolated by altering the temperature of the thermosensitive surface, thereby releasing the enriched ECM from the thermosensitive surface. In a particular aspect, the enriched ECM is attached to the scaffold before it is released from the thermosensitive surface. In another embodiment, the enriched ECM is released from the thermosensitive surface before it is attached to the scaffold.

In a certain aspect of this embodiment, the scaffold comprises a hydrogel.

### Exemplification

### Example 1

Skin is one of the most accessible tissues for experimental biomedical sciences, and cultured skin cells represent one of the longest-running clinical applications of stem cell therapy. However, culture generated skin-mimetic multicellular structures are still limited in their application by the time taken to develop these constructs in vitro and by their incomplete differentiation. The development of a functional dermal-epidermal junction (DEJ) is one of the most sought after aspects of cultured skin, and one of the hardest to recreate in vitro. At the DEJ, dermal fibroblasts and epidermal keratinocytes interact to form an interlinked basement membrane of extracellular matrix (ECM), which forms as a concerted action of both keratinocytes and fibroblasts. Successful formation of this basement membrane is essential for “take” and stability of cultured skin autografts.

Interactive matrix production by mono- and co-cultures of primary human keratinocytes and fibroblasts was studied in an attempt to improve the efficiency of basement membrane production in culture using mixed macromolecular crowding (mMMC). Resulting ECM were enriched in deposition of collagens I, IV, fibronectin and laminin 332 (laminin 5) and also in collagen VII, the anchoring fibril component. The in vitro data point to fibroblasts, rather than keratinocytes, as the major cellular contributors of the DEJ, as fibroblasts produced and deposited collagen VII in comparison to keratinocytes. In addition, decellularized fibroblast ECM stimulated production and deposition of collagen VII by keratinocytes, over and above that of keratinocyte monocultures. In confrontation cultures, keratinocytes and fibroblasts showed spontaneous segregation and demarcation of cell boundaries by DEJ protein deposition. Finally, mMMC was used in a classical organotypic co-culture protocol with keratinocytes seeded over fibroblast-containing collagen or fibrin gels. Applied during the submerged phase, mMMC was sufficient to accelerate the emergence of collagen VII along the de novo DEJ, together with stronger transglutaminase 2 activity in the neoepidermis. These findings corroborate the role of fibroblasts as important players in producing collagen VII and inducing collagen VII deposition in the DEJ, and that macromolecular crowding leads to organotypic epidermal differentiation in tissue culture in a significantly condensed time frame.

### Materials and Methods

a) Isolation of Primary Fibroblasts and Keratinocytes from Human Skin

Human dermal fibroblasts and human primary keratinocytes were isolated from normal human skin obtained from surgical waste skin remnants, with full local ethical approval. Skin was cleaned with ethanol and soaked in sterile PBS; excess fat was trimmed off using sterile surgical scissors. The skin was then incubated in 2× anti-mycotic solution (Sigma-Aldrich, Singapore) for 15 minutes at room temperature, followed by two washes in PBS. The skin was then cut into 1 cm×1 cm cubes and immersed in 2.4 U/ml Dispase solution (Roche, Singapore) for 16 hours at 4° C. The epidermis was then separated from the dermis by peeling it away with sterile forceps, and the incubated in 0.125% trypsin for 15 minutes at 37° C. before filtering through a 100 μm Nylon cell strainer (BD Falcon, BD Biosciences, USA) to remove tissue fragments. The resulting cell suspension was pelleted and resuspended in CNT-57 (Cell-NTec, Switzerland) or Keratinocyte Serum-Free Media (KSFM, Invitrogen, Singapore). After separation from the epidermis, the dermis was cut into smaller cubes and placed in a T-75 flask to generate explant cultures: fibroblasts began to migrate out from the explants after a few days.

### b) Cell Culture and Macromolecular Crowding

Fibroblasts were cultured in fibroblast medium (FM) made up of Dulbecco's modified Eagle's medium (DMEM; Invitrogen, Singapore) supplemented with 10% fetal bovine serum (FBS; Invitrogen, Singapore) and antibiotics; 100 U/mL penicillin (GIBCO-Invitrogen, Singapore) and 100 U/mL streptomycin (GIBCOInvitrogen, Singapore). Culture medium was changed every 2 days. Human primary keratinocytes were cultured in CNT-57 or KSFM. In addition, a keratinocyte cell line was used, NEB1.K14 wt-GFP, cultured in RM+ media containing Dulbecco's Modified Eagle Medium (DMEM; Invitrogen, Singapore), Ham's F12 (Invitrogen, Singapore) supplemented with 10% fetal bovine serum (FBS; Invitrogen, Singapore), antibiotics (100 U/mL penicillin (GIBCO-Invitrogen, Singapore) and 100 U/mL streptomycin (GIBCOInvitrogen, Singapore)), 0.4 μg/ml hydrocortisone, 5 μg/ml insulin, 1.8×10⁻⁴ M adenine, 10 ng/ml epidermal growth factor, 5 μg/ml transferrin and 2×10⁻¹¹ M lodothyronine. All cell cultures were maintained in a 37° C. humidified incubator with 5% CO₂.

A Ficoll™ cocktail consisting of 37.5 mg/ml of Ficoll™ 70 and 25 mg/ml of Ficoll™ 400 (Fc 70/400; SciMed Asia, Singapore) was used for mixed macromolecular crowding as described earlier (Chen C., Loe F., Blocki A., Peng Y., Raghunath M. Applying macromolecular crowding to enhance extracellular matrix deposition and its remodeling in vitro for tissue engineering and cell-based therapies. Adv. Drug Delivery Rev. 63(4-5), 277, 2011; Ang X. M., Lee M., Blocki A., Chen C., Ong S. L., Asada H., Sheppard A., Raghunath M. Macromolecular crowding amplifies adipogenesis of human bone marrow-derived MSCs by enhancing the pro-adipogenic microenvironment. Tissue Engineering, 2013, epub ahead of print). Briefly, cells were seeded onto the culture dish and allowed to attach overnight. The next day, culture medium was replaced with medium containing the macromolecular crowders together with ascorbic acid and cultured for 6 days with medium changes every 2 days.

To generate co-cultures, equal numbers of keratinocytes and fibroblasts were seeded together in a single well of a 24-well plate. To differentiate between keratinocytes and fibroblasts, NEB1.K14 wt-GFP keratinocytes were used, as they could be distinguished from the fibroblasts by their green fluorescent keratin filament network.

### c) Preparation of Decellularized Extracellular Matrix

Cells were grown in either non-crowded or crowded conditions prior to removing the cells to generate decellularized matrix. Cells were seeded onto 24-well plates and left to attach for 24 hours. Culture medium was then removed and new medium, with or without crowders, was added and cell cultures were maintained for 6 days, with media change every 2 days. Cell monolayers were decellularized using 0.5% sodium deoxycholate (Prodotti Chimici E Alimentari, S.P.A, Italy). Briefly, cell layers were washed twice with PBS, and 230 μl of sodium deoxycholate was added to the cell monolayers and left for 10 minutes on ice to lyse the cells. Lysates were then aspirated and the process was repeated two more times. After removing all cells, the remaining extracellular matrix preparations obtained were washed twice with distilled water and air-dried for 15 minutes in a laminar flow hood. Extracellular matrix preparations are referred to in the text as follows: f-mat=fibroblast-derived extracellular matrix, k-mat=keratinocyte-derived extracellular matrix, and extracellular matrices derived from co-cultures of fibroblasts and keratinocytes=co-mat. Fibroblasts were seeded on top of k-mat and left to adhere overnight. Similarly, keratinocytes were seeded on f-mat and left to adhere overnight. Colony forming assay was performed as previously described (Barrandon Y. and Green H. Three clonal types of keratinocyte with different capacities for multiplication. Proc. Natl. Acad. Sci. 84, 2302, 1987).

### d) Immunocytochemistry and Histochemistry

Cell layers and decellularized extracellular matrices were fixed with either methanol or 3% paraformaldehyde (PFA). Non-specific binding of antibodies was blocked by incubating with 3% bovine serum albumin (BSA) or 10% goat serum for 30 minutes. Primary antibody was then added for 90 minutes at room temperature. Primary antibodies include rabbit anti-collagen I (Abcam, dilution 1:100), mouse anti-collagen IV (NovoCastra, PHM-12 clone, dilution 1:100), mouse anti-collagen VII (clone LH7.2) mouse anti-fibronectin (Abcam, dilution 1:100), mouse anti-heparan sulfate (Abcam, dilution 1:100), mouse anti-collagen XVII (Abcam, dilution 1:100) and mouse anti-vimentin (Chemicon, V9 clone, dilution 1:100). Secondary antibodies (AlexaFluor 594 goat anti-rabbit, AlexaFluor 594 goat anti-mouse, Alexa Fluor 488 chicken anti-rabbit and AlexaFluor 488 goat anti-mouse; Molecular Probes) were then added for 30 minutes at room temperature. Nuclei were counterstained with 4′-6-diamidino-2-phenylindole (DAPI). Coverslips were mounted onto a glass slide using Hydromount mounting media (National Diagnostics, USA). Samples were left to air dry for 24 hours before visualization using an inverted Olympus fluorescent microscope or a Zeiss Confocal microscope. The transglutaminase assay was conducted as previously described (Raghunath M., Hennies H. C., Velten F., Wiebe V., Steinert P. M., Reis A., Traupe H. A novel in situ method for the detection of deficient transglutaminase activity in the skin. Arch. Dermatol. Res. 290, 621, 1998).

### e) Histology

Organotypic cultures were cut into pieces and fixed separately, one snap frozen and one in 10% neutral buffer formalin, and processed into wax blocks for tissue sectioning. For frozen tissue, blocking and subsequent primary and secondary antibody incubation could be performed immediately. For formalin fixed paraffin embedded sections, the sections were subjected to a routine histological protocol for H&E staining.

### f) Immunoblotting

Cell layers (seeded on 6-well plates) were washed twice in phosphate buffered saline (PBS), then scraped into 100 μl lysis buffer and then centrifuged at 14000 rpm for 30 minutes at 4° C. The supernatant was collected, mixed with sample buffer and heated for 10 minutes at 95° C. After spinning down to collect condensation water, a final volume of 200 was loaded onto pre-cast 3-8% NuPAGE gels (Invitrogen, Singapore).

The concentration of proteins in the samples were determined by Pierce BCA Protein Assay Kit (ThermoScientific, USA) and equal amounts of protein were loaded onto SDS-PAGE gels (Invitrogen, Singapore) as confirmed by the actin blots. Separated proteins were transferred to nitrocellulose membranes (Biorad, Germany) and blocked with 5% milk powder for 1 hour at room temperature. Primary antibody used was mouse anti-collagen VII (clone LH 7.2, 1:1000). Secondary antibody used was goat anti-mouse-HRP. After washing with PBS-Tween, membranes were incubated with ECL detection reagents (GE Healthcare, Singapore) and chemiluminescence detected on light sensitive film (GE Healthcare, Singapore).

g) Interference Reflection Microscopy (IRM) Combined with Fluorescence Microscopy

Image acquisition was performed by using a Confocal Laser Scanning Microscope (LSM510, Zeiss, Germany) with an EC Plan-Neofluar 40×/1.30 Oil objective. The pinhole was set up at 74 μm. The filters used were LP 505 for the IRM channel and BP 575-615 IR for the fluorescence red channel. The beam splitters used were NT 80/20 for the IRM channel and HFT 405/488/561, NFT 565, Plate for the fluorescence red channel. The Lasers used were DPSS 561-10 (wavelength 561 nm) at 1.1% power for the fluorescence red channel and HeNe633 (wavelength 633 nm) at 5.0% power for the IRM channel.

### h) 3D Organotypic Co-Cultures

Collagen Gel Preparation:

Keeping all solutions on ice; 10 ml of collagen solution (rat tail collagen type I (BD Biosciences, USA), suspended in acetic acid) was mixed with 1 ml DMEM followed by 0.5 ml 1M NaOH for neutralization. 0.5 ml of DMEM containing 1,200,000 fibroblasts was added. 2 ml of the collagen-fibroblast suspension was loaded into a cell culture insert (BD Biosciences, USA) in each well of a 6-well plate and the plate transferred to 37° C. to allow the collagen to gel for 1 hour.

Fibrin Gel Preparation:

Tisseel (Baxter) gel was prepared according to the manufacturer's instructions. Fibroblasts were incorporated into the fibrin gel solution before it solidified at 37° C. Fibrin gels were used to control for the presence of interference from any preformed collagen fibres.

After the collagen or fibrin gel had solidified, FM was added to the fibroblast-containing gel in the cell culture insert. After 24 hours, FM was replaced with FM containing the Fc 70/400 crowder cocktail and ascorbic acid. On the third day, the medium was aspirated and keratinocytes were seeded on top of the collagen or fibrin gel. After 3 hours, the keratinocytes have attached to the gel, after which CNT-57 or KSFM was added in the cell culture insert. The following day, the medium was replaced with CNT-57 or KSFM containing the Fc 70/400 crowder cocktail and ascorbic acid. After 1 week of submerged culture, the keratinocytes have formed a confluent layer on top of the gel. Cell culture inserts are then raised to the air-liquid interface using deep well plates (BD Biosciences, USA) to facilitate keratinocyte stratification and differentiation. After 3-5 weeks, the mature organotypic cultures are harvested for analysis.

### i) Electron Microscopy

Organotypic skin co-cultures were fixed in 2.5% glutaraldehyde (in PBS) for 72 hours. Samples were then washed in PBS and cut into small pieces (1 mm³) Post-fixation was carried out in 1% osmium tetroxide, pH 7.4, for 1 hour at room temperature. Samples were then washed in distilled water and dehydrated through an ascending ethanol series. Samples were infiltrated, embedded in araldite resin and left to polymerize at 60° C. for 24 hours.

### Results

### (I) Macromolecular Crowding Enhances the Deposition of ECM Components Synthesized by Fibroblasts, Keratinocytes and a Fibroblast-Keratinocyte Co-Culture In Vitro

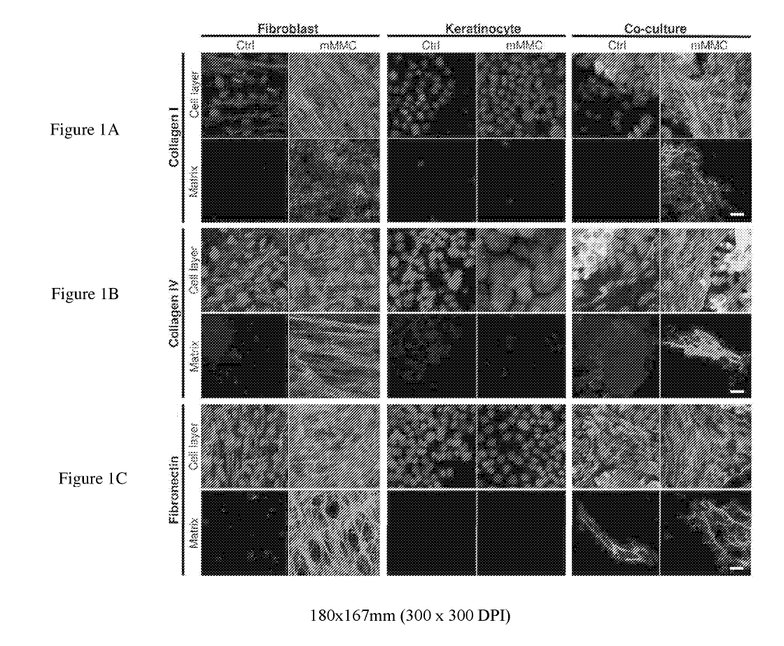

Upon the addition to the culture medium of a Ficoll™ 70/400 macromolecular crowding mixture, collagen type I, IV and fibronectin production by fibroblasts was dramatically enhanced in 6 days as compared to uncrowded controls. Decellularization of the mMMC-treated fibroblast cell layer yielded an enriched matrix on the tissue-culture polystyrene surface (TCPS) (FIG. 1), demonstrating a significantly increased amount of matrix (containing collagen type I, IV and fibronectin) deposited by the fibroblasts, as compared to control cultures in which minimal matrix was detected.

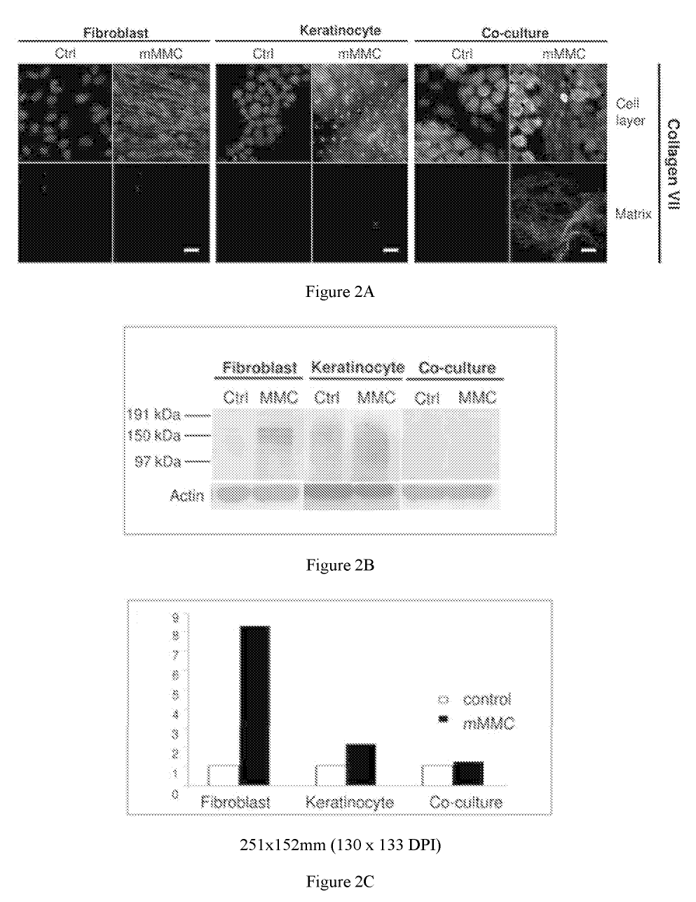

As keratinocytes do not synthesize collagen I, mMMC only enhances intrinsic deposition of various amounts of fibronectin and in particular DEJ components such as collagen type IV (FIG. 1) (Prunieras M., Regnier M., Fougere S., Woodley D. Keratinocytes synthesize basal-lamina proteins in culture. J. Invest. Dermatol. 81(1Suppl), 74s, 1983). The highest level of collagen type I, IV and fibronectin deposition was observed in confrontation cultures of fibroblasts and keratinocytes, cultured under mMMC suggesting a stimulatory role of keratinocytes on fibroblasts. Although fibroblasts and keratinocytes were initially seeded as a heterogeneously mixed cell suspension, the two cell types demixed and segregated into distinct populations. Keratinocyte colonies could be distinguished from fibroblasts by the green fluorescence of the GFP-coupled keratin-14 (K14) filaments. Comparing the three cell seeding situations and the resulting ECM after decellularization, it became obvious that although mMMC-treated fibroblasts produce a rich ECM, they deposited significantly more in co-culture with keratinocytes. In our hands, collagen VII was synthesized predominantly by fibroblasts and to a lesser extent by keratinocytes. While there was a small amount of collagen type VII produced by both cell types in vitro, mMMC dramatically enhances this production and deposition (FIG. 2A). In particular, strong collagen type VII staining was noted in fibroblast cultures crowded with Fc 70/400. In contrast, a co-culture of fibroblasts and keratinocytes under the same conditions but without mMMC treatment failed to yield significant amounts of collagen type VII. Whole cell lysates also show an increased amount of collagen type VII production after mMMC exposure when compared to uncrowded controls (FIGS. 2B-C) suggesting a rich pericellular matrix.

(II) Fibroblast-Derived Matrices (f-Mat) Induce Keratinocytes to Deposit More Collagen Type VII In Vitro

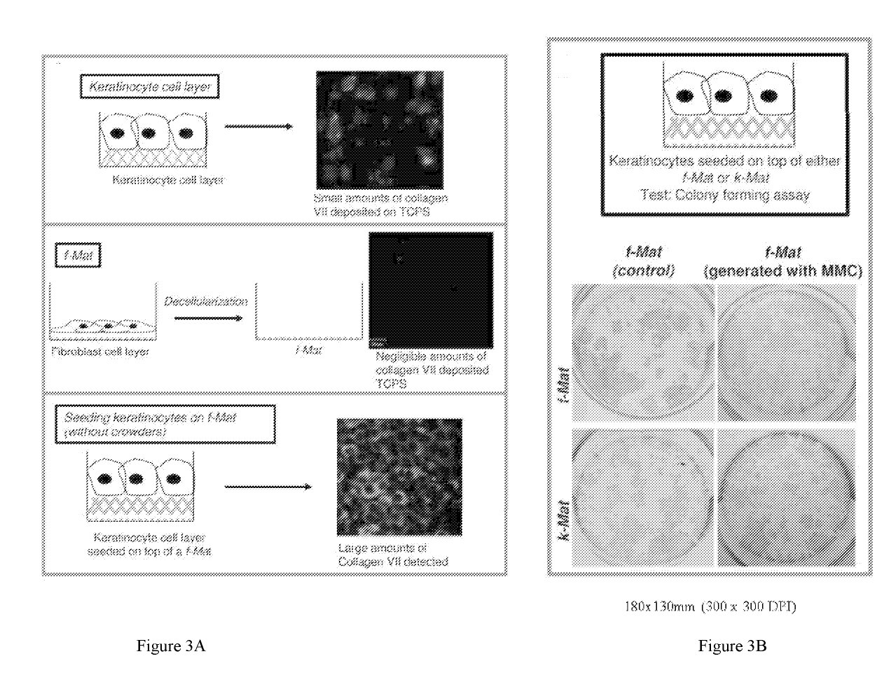

Upon seeding onto a fibroblast-derived matrix (f-mat), keratinocytes were able to synthesize a larger amount of collagen type VII than keratinocytes cultured only on TCPS. This points to an effect of the fmat on the keratinocytes and the capacity of this matrix to stimulate ECM production in cells with which it comes into contact (FIG. 3). Colony forming assays showed that keratinocytes were able to adhere and form colonies on f-mat and k-mat. Keratinocytes were also induced to produce significant amounts of collagen type IV when they were seeded on a previously Ficoll™ 70/400-crowded f-mat (data not shown).

(III) Interference Reflection Microscopy Demonstrates Larger Amount of ECM Deposited Under mMMC.

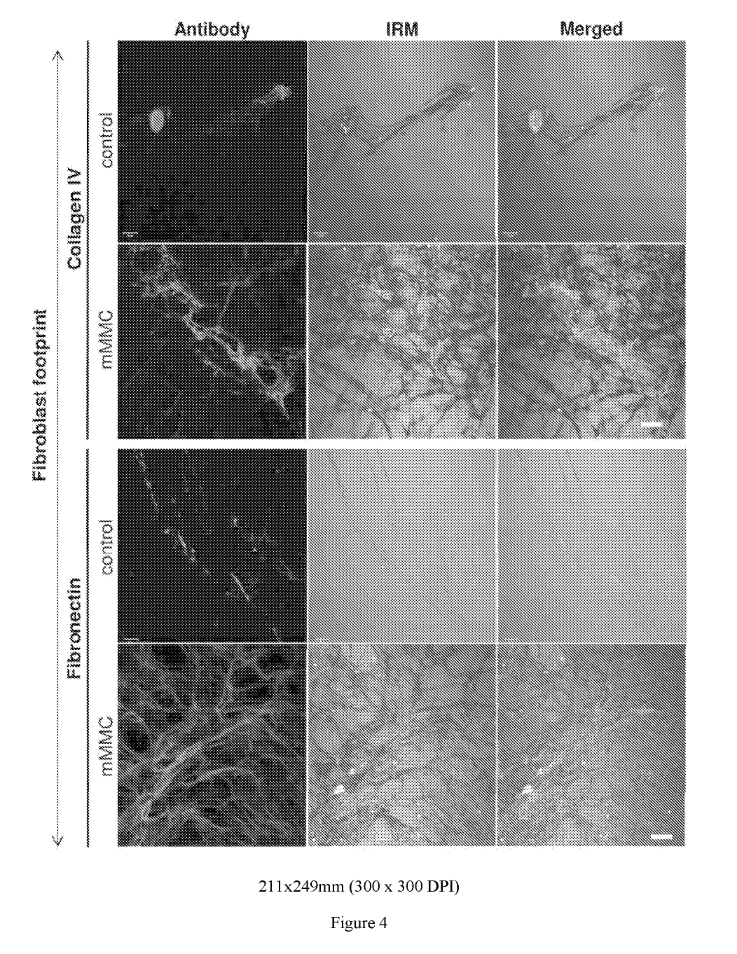

Interference reflection microscopy was applied to visualize the whole extracellular matrix deposited by and in contact with the glass coverslip. All the matrix components which were in contact with the glass coverslip generated a dark print by IRM (black area). Here, fibroblasts were cultured under crowded and non-crowded conditions and subsequently these cell layers were removed to reveal the underlying matrix (fibroblast footprint). Upon antibody staining with collagen IV, uncrowded cultures showed only patches of collagen IV. In crowded fibroblast cultures, the amount of collagen IV increases significantly (FIG. 4; collagen IV antibody). With IRM, the whole extent of the extracellular matrix could be detected. Comparing between crowded and control fibroblast footprints, it is evident that crowding causes fibroblasts to deposit more matrix extracellularly as is seen when comparing the IRM-crowded and IRM-control images.

The deposition of fibronectin was also studied and it was noted that the extracellular matrix is composed largely of this component (FIG. 4; IRM-fibronectin antibody merge). Mixed macromolecular crowding increases this production and deposition by fibroblasts as seen in both antibody and IRM images.

### (IV) Macromolecular Crowding Enhances the Deposition of Collagen Type VII in the Dermal-Epidermal Junction of Organotypic Co-Cultures In Vitro

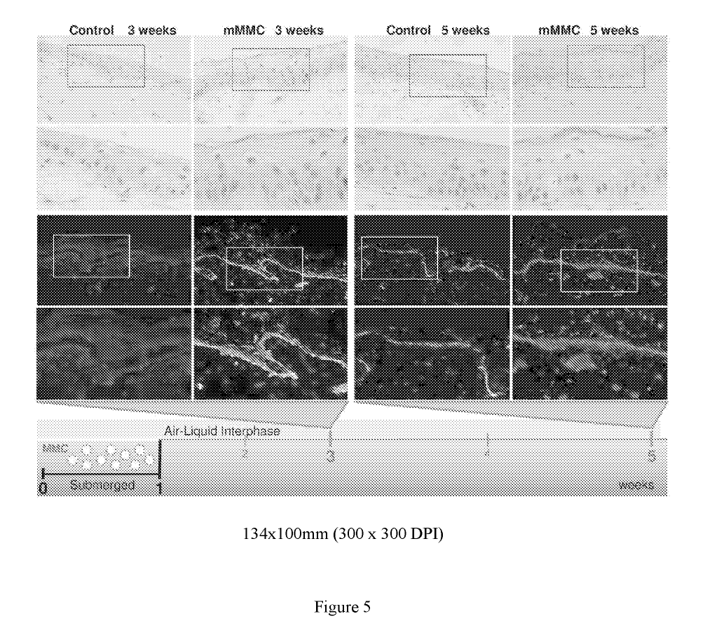

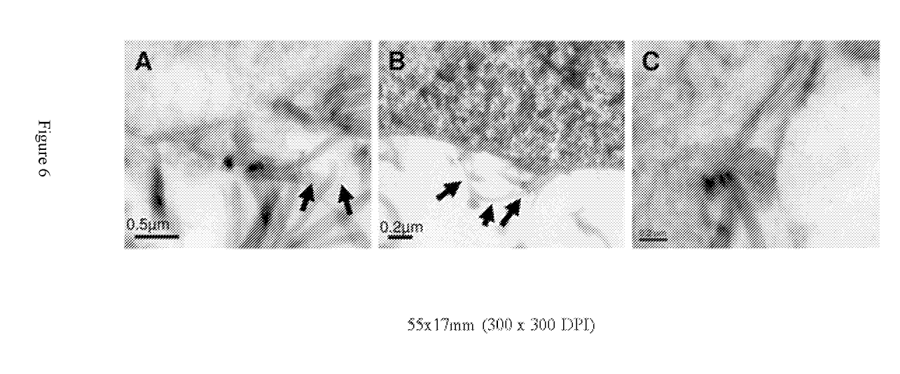

The ECM deposition enhancement by macromolecular crowding was evaluated in organotypic cultures by H&E staining as well as by antibody staining of specific proteins. As a starting scaffold to host dermal fibroblasts, rat tail collagen type I was used as a dermal substitute. Fibrin gel was also used as a dermal scaffold to allow for quantitation of de novo synthesized and deposited collagen type I. In addition to observing the three-dimensional pattern of ECM enhancement, the organotypic co-cultures also allow to study the stratification status of the epidermis. mMMC was evaluated as a potential accelerator of DEJ maturation and epidermal stratification that was applied during the first week, the immersion phase. Two weeks after the constructs had been lifted to the air-liquid interphase (meaning a total age of 3 weeks) the de novo dermal-epidermal junction showed under mMMC, strong collagen VII which was negligible in non-crowded controls (FIG. 5). Only 5-week non-crowded controls started showing some presence of collagen VII expression along the neo-DEJ, albeit sporadic and still reduced compared to the 5-week old skin equivalent cultured with mMMC. Interestingly, anchoring fibrils were observed in the neo-DEJ of 3-week old skin equivalents generated with mMMC (FIG. 6A-B; arrows), but not in control cultures, pointing to the probability that the increased amount of collagen VII secreted under crowded conditions has the capability to form anchoring fibrils in vitro.

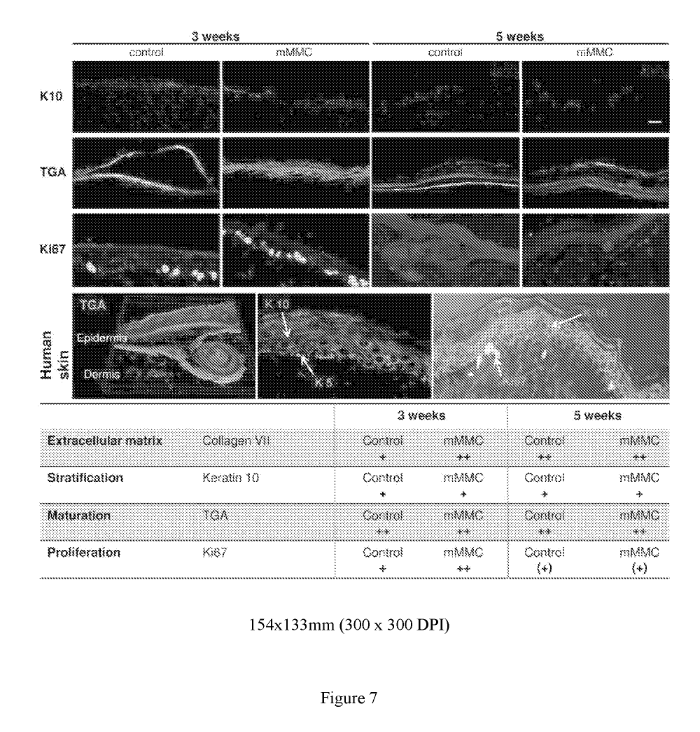

Also, epidermal maturation was improved by mMMC. After 3 weeks, a mature pluristratified epidermis under mMMC was observed which was absent in non-crowded controls (FIG. 5). After 5 weeks of culture, both crowded and non-crowded skin equivalents showed a stratified epidermis. Organotypic culture maturity was further assessed by proliferation and stratification markers, including Ki67, keratin 10 and transglutaminase activity (FIG. 7). Keratin 10 expression was present in the epidermal layers of all organotypic cultures showing stratification of skin equivalents under the various conditions. While transglutaminase activity was present in the epidermal layers of all organotypic cultures, it was observed that in 3-week old mMMC organotypic cultures, transglutaminase activity was more evenly expressed in the whole epidermal layer. Ki67 positive cells were observed to be most abundant in 3-week old mMMC organotypic cultures as compared to 3-week old controls. 5-week old mMMC organotypic cultures showed negligible amounts of Ki67 positive cells suggesting an extremely low proliferative capacity of the epidermal layer.

### DISCUSSION

As shown herein, two different cell types, fibroblasts and keratinocytes cooperate to build the dermal-epidermal junction in vitro at the two-dimensional (2D) and three-dimensional (3D) level. This cellular cooperation is based on cell-matrix reciprocity effects. In order to dissect these effects, a novel technology in tissue engineering, macromolecular crowding, a system based on the introduction of carbohydrate-based macromolecules in a culture system was applied. The resulting excluded volume effects drive enzymatic processes that control ECM processing and also supramolecular assembly. A particularly intriguing observation was that it was possible under mMMC to affect deposition of collagen VII in keratinocyte monocultures to the extent, that after removal of these cells, a footprint remained. This is believed to be the first report on the successful in vitro deposition of collagen VII, the major component of anchoring fibrils and essential guarantors of dermo-epidermal cohesion. In confrontation cultures of fibroblasts and keratinocytes, a segregation of cell types was observed, as well as a demarcation of the boundaries between both cell types characterized by the deposition of collagens IV and VII. The deposition of collagen VII allowed an investigation of the effects of fibroblast matrices (fmats) on keratinocytes and vice versa. mMMC technology helped to show that dermal fibroblasts are the main producers and depositors of collagen type VII.

MMC initially augments the production of ECM as it mimics more closely the in vivo environment. Once ECM is deposited, a solid phase microenvironment has arisen in which the producing cells find themselves embedded. This ECM contains biochemical cues that fuel dynamic cell-matrix reciprocity. After decellularization, crucial information is retained in these matrices and thus is still available to cells that are freshly seeded onto these matrices. Here, a vast difference became apparent between ECM that had been generated in standard culture or under mMMC. Utilizing the cell-derived matrices (f-Mat, k-Mat, co-Mat) for bio-engineering applications is advantageous over commercial coatings, as they are less denatured, fully cell-type specific, fully processed, secreted and deposited onto the culture surface containing a full portfolio of ligands (such as proteoglycans and growth factors). Thus, the mMMC-generated ECMs, here the Mats (f-Mat, k-Mat, co-Mat) represent highly complex mixtures of ECM molecules produced and processed by the cell, as opposed to an artificial layer of a single basement membrane component commonly used as coating. This would predict a much more physiological in vivo extracellular environment. Interestingly, keratinocytes seeded on f-Mat were induced to produce and deposit collagen type VII, which they do not normally do in such amounts when cultured on TCPS alone, pointing to the effect of matrix reciprocity.

Taking these observations to a tissue equivalent level, MMC was introduced to three-dimensional organotypic cultures and found to be an impressive improvement of both expression of DEJ proteins and maturation of the epidermis. Current organotypic culture protocols involve at least 2 weeks at the air-liquid interface with a total culture time of 4-5 weeks. Using mixed macromolecular crowding in a modified organotypic culture protocol, the time needed to generate a skin equivalent was reduced from 5 weeks to 3 weeks, involving 1 week of submerged culture in the presence of MMC followed by a 2 week air-liquid interphase. This generated an organotypic skin equivalent with a pluristratified epidermis as well as an enhanced basement membrane, in particular, the deposition of collagen type VII in the DEJ is enriched, as compared to the uncrowded control which show a spotty and discontinuous expression of collagen type VII in the DEJ. Ultrastructural investigations suggested an early stage of anchoring fibril appearance in mMMC treated organotypic cultures.

As macromolecular crowding works as an amplifier of enzymatic activity, the role of matrix metalloproteinases, drivers of remodeling, should also be predictably enhanced under mMMC. A faster dissolution of fibroblasts seeded on collagen gels under crowded conditions was observed (not shown) which ties in with observations of dissolving chondrocyte pellets under MMC. Recent findings confirm that MMP2 activity is increased and more closely associated with the pericellular matrix under MMC. While from the physiological point of view this will open highly interesting avenues to study tissue remodeling under MMC, biotechnological considerations would suggest to use MMC in selected time windows to preferably harness the anabolic phase of tissue growth. Here, the first week in submerged conditions was found to be optimal for accelerating the maturation of organotypic skin equivalents. These findings have major implications for the preparation of autografts for skin defects or organotypic skin constructs for pharmacological studies.

One of the biggest problems in autologous keratinocyte grafting is the time it takes to grow keratinocyte sheets or living skin equivalents. In the first case, the stabilization of the DEJ after grafting involves the cooperative formation of a neoepidermis and neo-dermis and it takes 120 days, at least in vivo, until the formation of sizeable anchoring fibrils are evident. As long as the DEJ is not well formed, the grafts are fragile. Transplanting pre-constructed skin equivalents with a pre-stabilized DEJ would be a desirable alternative to pure keratinocyte autografting. Culturing autologous co-cultures of fibroblasts and keratinocytes as skin equivalents, for example in a fibrin hydrogel, has long been the gold standard in skin grafting protocols. The current time required from harvesting of the cells to maturation of the skin equivalent can range from 4 to 8 weeks. Therefore, the method described herein of culturing fibroblasts and keratinocytes in a mixed macromolecular crowded hydrogel to generate an organotypic skin equivalent in a shorter time frame, while still maintaining a pluristratified epidermis and promoting mature DEJ formation, holds great potential in tissue engineering and wound healing.

### Example 2

Acellular matrices that are rich in extracellular matrix (“enriched ECM” or “eECM”) have potential for use as scaffolds for secondary cell seeding, for example of embryonic stem cells. However, current cell culture techniques for generating such matrices yield a fragile layer of eECM on the culture surface, which is challenging to handle and/or transfer for biomedical applications. Accordingly, there is a need to develop new methods for the production of eECM that would make a more robust eECM.

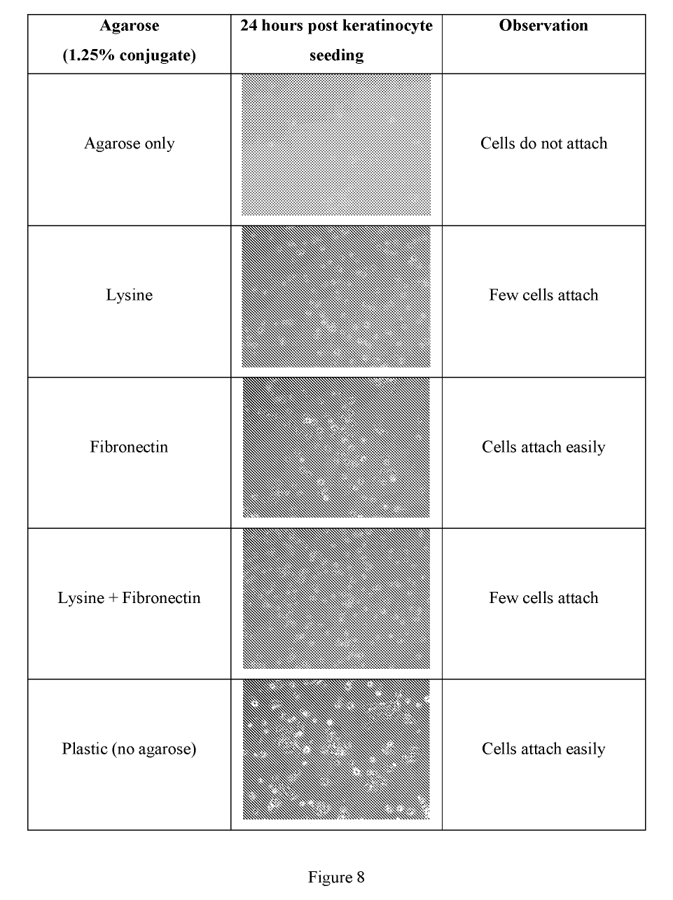

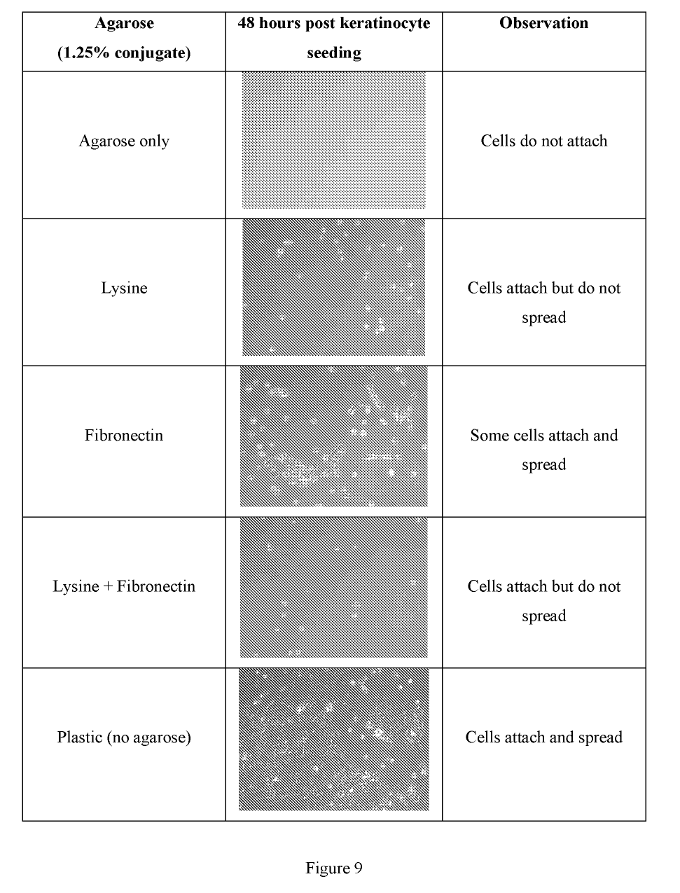

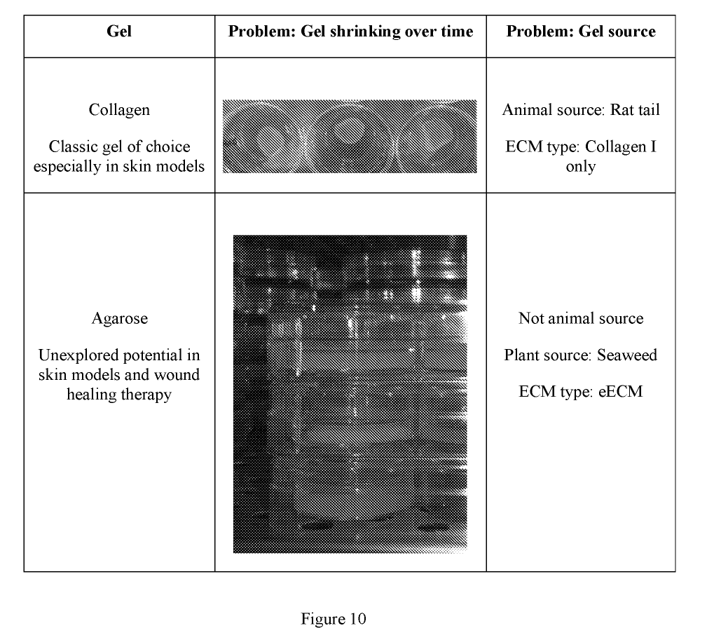

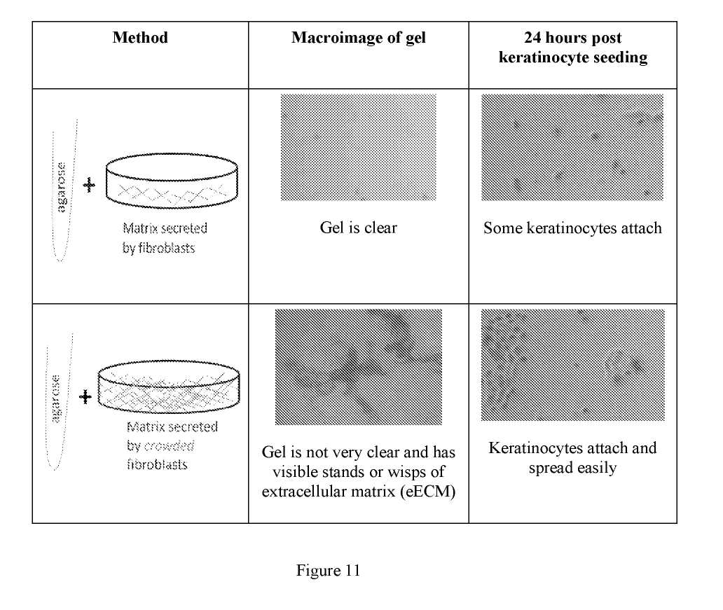

An agarose-eECM gel was developed, which could be customized to fit different wounds to improve skin wound healing. Agarose was chosen as the preferred gel for a number of reasons. For example, agarose is readily available, inexpensive and non-xenogeneic. Nonetheless, agarose had been an unexplored option as a scaffolding material for use in skin wound healing. As gel stiffness may affect cell adhesion and proliferation, it was important to optimize a working concentration of agarose. Moreover, as mammalian cells attach poorly to agarose, commercially available compounds (collagen IV, fibronectin, poly-L-lysine) were screened as potential agarose conjugates to enhance cell adhesion on the gel. Composite gels were developed and compared with an agarose-eECM gel. The data provided herein has shown the possibility of creating and using an agarose-eECM gel as a scaffold for eECM production (see Table 1 and FIGS. 8-11), moving towards a xenogeneic-free system that involves cell-derived molecules in contrast with commercially produced synthetics.

TABLE 1
Optimizing the Agarose Gel Concentration

| Percentage | Observation | |
|---|---|---|
| of agarose | Handling | Gelation |
| 0.5% | Too soft | Cells do not readily |
| | | attach |
| 1% | Soft | Cells attach after |
| | | some time |
| 1.25% | Optimal for handling | Speed of gelation is |
| | | slow enough to add |
| | | other components |
| 2.5% | Easy to handle | Solidifies fast |
| 5% | Viscous and difficult to handle | Solidifies fast |
| 10% | Too viscous to handle | Solidifies fast |

### Example 3

Decellularized ECMs derived from different cell types were charcaterized by FESEM (field emission scanning electron microscopy) analysis, which provides an ultrastructural view of the cell-derived matrices. The cell-derived matrices were prepared from fibroblasts (f-Mat), keratinocytes (k-Mat) or a co-culture of fibroblasts and keratinocytes (co-Mat), as follows:

f-Mat: extracellular matrix obtained from decellularizing a fibroblast cell layer which have been cultured with macromolecular crowders;

k-Mat: extracellular matrix obtained from decellularizing a keratinocyte cell layer which have been cultured with macromolecular crowders;

co-Mat: extracellular matrix obtained from decellularizing a co-culture of fibroblasts and keratinocytes, which have been cultured with macromolecular crowders.

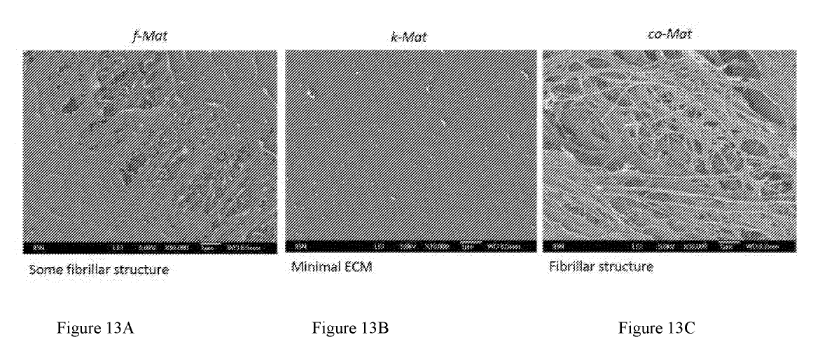

co-Mat matrices had the most fibrillar structure, resembling that of human dermal collagen (FIG. 13C). K-mat matrices had the least amount of fibrillar structure (FIG. 13B), while f-mat matrices had an intermediate fibrillar structure (FIG. 13A).

Comparing the co-Mat FESEM images with FESEM images of Matrigel, collagen I-fibrin gels and dermal collagen in published sourcesit revealed that the co-Mat matrices have a structure similar to dermal collagen. This structure is also comparable with gels such as collagen I-fibrin gels. An advantage of the co-Mat ECM over Matrigel, which is derived from mouse sarcoma cells, is that the co-Mat matrix is xenogene-free.

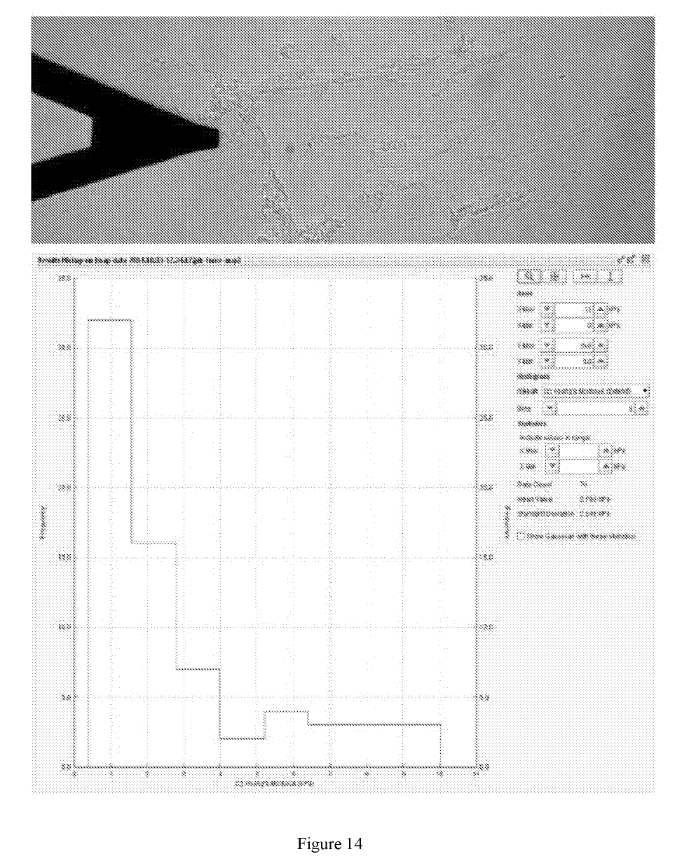

AFM (atomic force microscopy) was also utilized to evaluate the mechanical properties of the cell-derived matrices. It was determined that co-Mat, generated using macromolecular crowders, was of significant thickness as compared to controls (without MMC). Furthermore, the stiffness of these matrices was approximately 1 kPa, which corresponds to a soft biomaterial (FIG. 14).

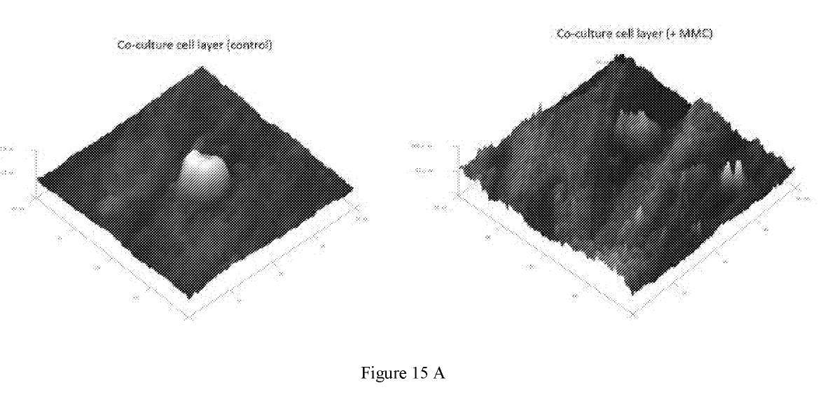

In addition, preliminary data on the scanning of cell layers (before decellularization) revealed ECM enhancement with MMCs relative to cultures without MMCs (FIG. 15A).

A depth color-coding system was applied to clearly illustrate the thickness of the cell-derived matrix/Mats. The thickness of f-Mat was calculated to be approximately 4.8 μm. With this imaging technique, a clearer picture of the thickness and distribution of the cell-derived matrices was achieved (FIG. 15B).

### Example 4

The functionality of the cell-derived matrices as a stem cell niche was tested using a colony forming assay. Keratinocytes were grown on these matrices to assess the colony forming efficiency.

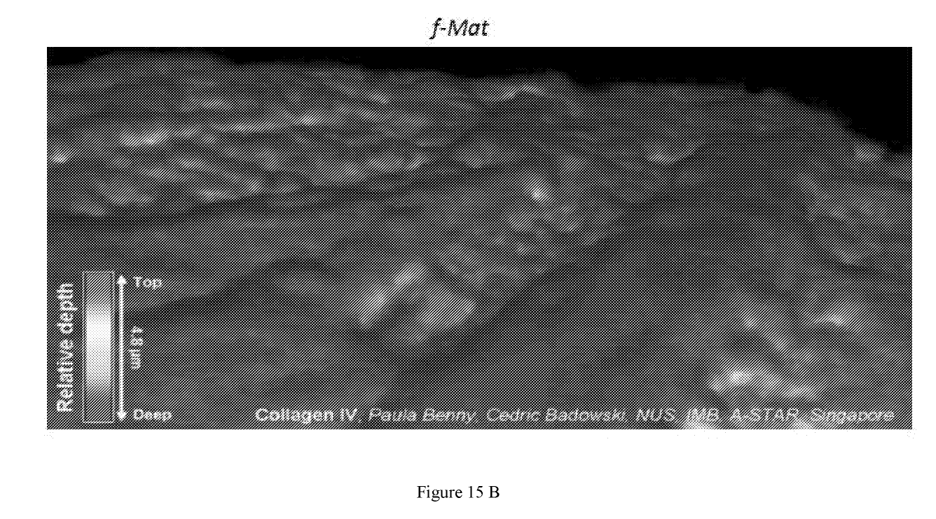

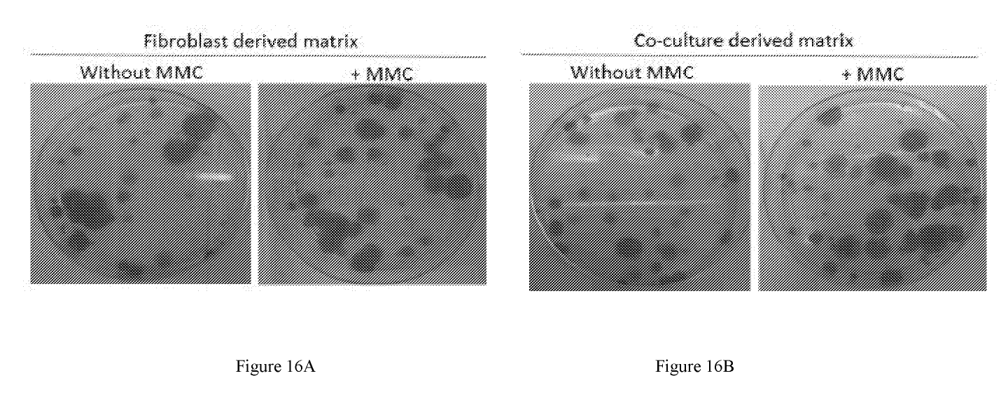

It was observed that f-Mat and co-Mat matrices generated with macromolecular crowders were able to sustain many more and larger colonies as compared to Mats generated without macromolecular crowders (FIGS. 16A and 16B). It was also observed that more cells were rescued when seeded on a co-Mat matrix when cultured with MMCs (FIG. 16B).

### Example 5

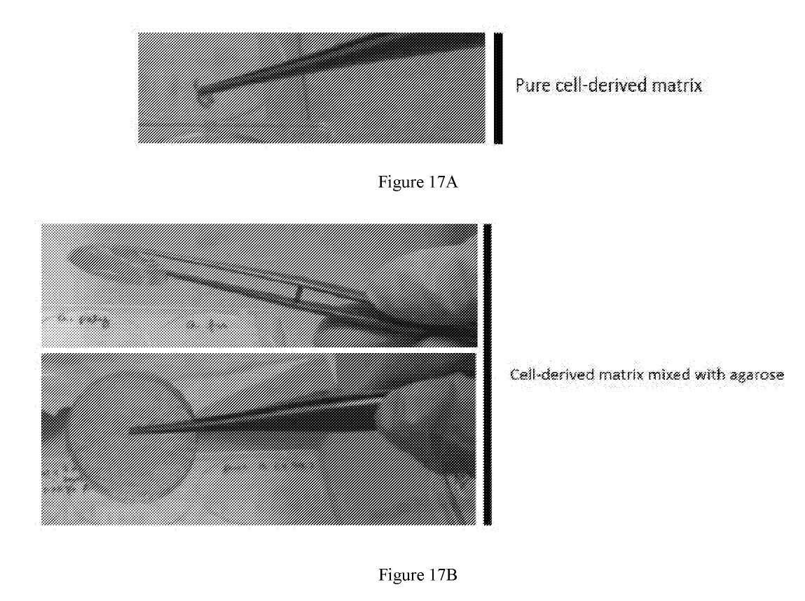

Various ECM-gels derived from decellularised crowded cell layers were mixed with different hydrogels to improve handling of the matrices so that they can be manipulated and transferred more readily (e.g., from an original culture vessel, such as a plate or dish, to another location, such as another culture vessel or wound bed). These carrier gels can make the ECM matrix material easier to manipulate without affecting the positive properties of the cell-derived matrix (see FIGS. 17A and 17B).

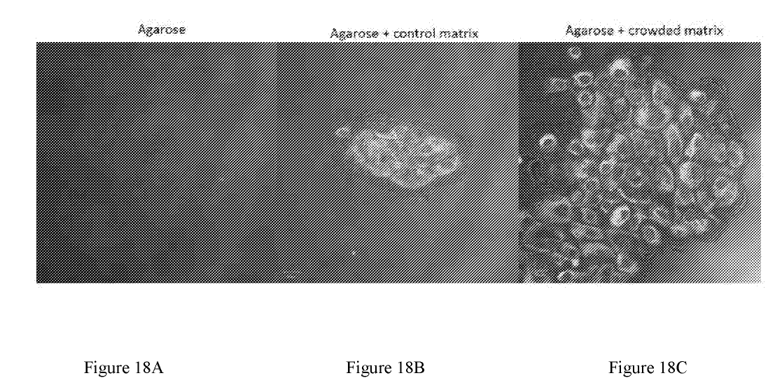

Several ECM-gels were made using different gel compounds, such as agarose, agarosepoly-lysine, alginic acid from brown algae and sodium alginate, with the cell-derivedmatrices/Mats. Preliminary data suggests that an agarose-ECM gel supports keratinocyte growth best (FIGS. 18A-C; blue nuclei; green-keratin; IRM).

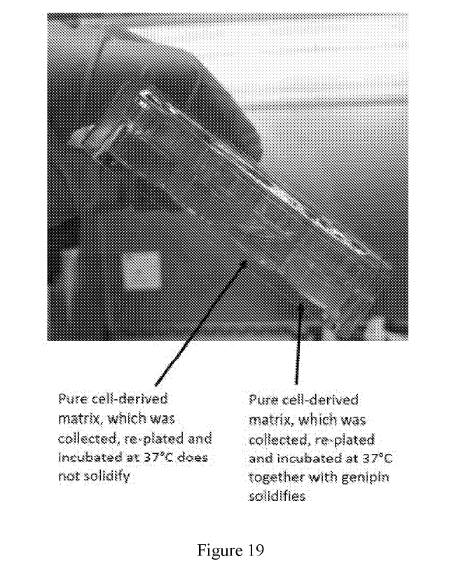

The effects of cross-linking agents, which could harden the pure cell-derived matrix, were also tested as an alternative to using a hydrogel. In particular, genipin, which is a cross-linker and a fruit extract, was incorporated into a cell-derived matrix to obtain a non-hydrogel ECM-gel (FIG. 19).

### Example 6

The potential for different ECM-gels to support cell growth and tissue/tissue mimetic formation was evaluated for a variety of cell types. Table 2 includes a list of cell types, gels, namely, skin keratinocytes, lung fibroblasts, kidney cells, endothelial cells and neurons, that were tested for biocompatibility with different ECM-gels.

TABLE 2
Biocompatibilites of Cell Types with ECM-gels.

| | | Bio- | |
|---|---|---|---|
| | | compat- | |
| Carrier Gel | Cell type | ibility | Conditions |
| Agarose | Skin keratinocyte | [symbol] | Gel only |
| | | [symbol] | Gel + uncrowded matrix |
| | | [symbol] [symbol] [symbol] [symbol] | Gel + Fc/crowded matrix |
| | Lung fibroblast | [symbol] | Gel only |
| | | [symbol] [symbol] [symbol] | Gel + Fc/crowded matrix |
| | Kidney cell | [symbol] | Gel only |
| | | [symbol] [symbol] | Gel + Fc/crowded matrix |
| | Endothelial cell | [symbol] | Gel only |
| | | [symbol] [symbol] | Gel + Fc/crowded matrix |
| | Neurons | [symbol] | Gel only |
| | | [symbol] | Gel + Fc/crowded matrix |
| Agarose- | Skin keratinocyte | [symbol] | Gel only |
| poly-lysine | | [symbol] | Gel + uncrowded matrix |
| | | [symbol] [symbol] | Gel + Fc/crowded matrix |
| | Lung fibroblast | [symbol] | Gel only |
| | | [symbol] | Gel + Fc/crowded matrix |
| | Kidney cell | [symbol] | Gel only |
| | | [symbol] | Gel + Fc/crowded matrix |
| | Endothelial cell | [symbol] | Gel only |
| | | [symbol] | Gel + Fc/crowded matrix |
| | Neurons | [symbol] | Gel only |
| | | [symbol] | Gel + Fc/crowded matrix |
| Alginic acid | Skin keratinocyte | [symbol] | Gel only |
| | | [symbol] | Gel + uncrowded matrix |
| | | [symbol] | Gel + Fc/crowded matrix |
| | Lung fibroblast | [symbol] | Gel only |
| | | [symbol] [symbol] | Gel + Fc/crowded matrix |
| | Kidney cell | [symbol] [symbol] | Gel only |
| | | [symbol] [symbol] [symbol] | Gel + Fc/crowded matrix |
| | Endothelial cell | [symbol] | Gel only |
| | | [symbol] [symbol] | Gel + Fc/crowded matrix |
| | Neurons | [symbol] | Gel only |
| | | [symbol] | Gel + Fc/crowded matrix |
| Sodium alginate | Skin keratinocyte | [symbol] | Gel only |
| | | [symbol] | Gel + uncrowded matrix |
| | | [symbol] | Gel + Fc/crowded matrix |
| | Lung fibroblast | [symbol] | Gel only |
| | | [symbol] | Gel + Fc/crowded matrix |
| | Kidney cell | [symbol] [symbol] | Gel only |
| | | [symbol] [symbol] [symbol] | Gel + Fc/crowded matrix |
| | Endothelial cell | [symbol] | Gel only |
| | | [symbol] [symbol] | Gel + Fc/crowded matrix |
| | Neurons | [symbol] | Gel only |
| | | [symbol] | Gel + Fc/crowded matrix |

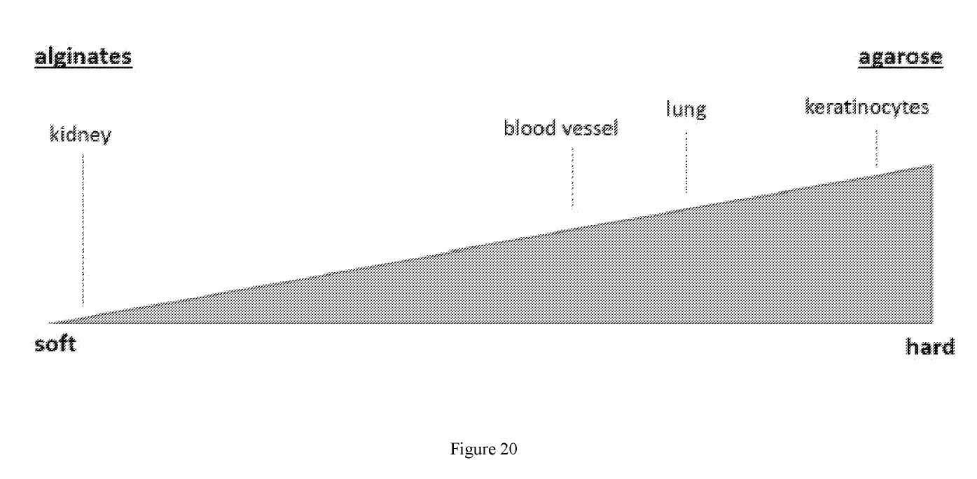

It was noted that alginic acid and sodium alginate tended to form softer gels as compared to agarose and agarose-poly-lysine (FIG. 20). This could explain why keratinocytes adhered better to agarose gels, while kidney cells adhered better to alginate gels. Without wishing to be bound by any one theory, it is believed that cells from organs exposed to higher mechanical stress, such as the skin, would adhere better to a harder substrate as compared to a softer one.

### Example 7

The use of UPCELL™ surfaces, which are made from a thermo-responsive polymer that allows non-enzymatic and non-mechanical harvest of surface proteins, to keep the cell-derived matrix as an intact sheet for future applications was tested. FIGS. 12B and 12C illustrate different ways of constructing an ECM-gel using UPCELL™ FIG. 12B shows a method for producing an ECM-gel with the cell-derived matrix concentrated on one surface. FIG. 12C shows generation of an ECM gel wherein the cell-derived matrix is distributed evenly throughout the gel.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of producing a tissue-mimetic construct having a basement membrane, comprising:
a) combining epithelial cells and stromal cells with one or more macromolecules having a hydrodynamic radius in the range of from about 2 nm to about 50 nm, thereby producing a mixed cell culture; and
b) maintaining the cell culture under conditions in which the epithelial cells and stromal cells proliferate and produce a basement membrane,
thereby producing a tissue-mimetic construct having a basement membrane.

2. The method of claim 1, wherein the tissue-mimetic construct is a skin-mimetic construct, a corneal tissue-mimetic construct, an oral mucosa-mimetic construct, a bladder tissue-mimetic construct, a liver tissue-mimetic construct, a pancreatic tissue-mimetic construct, a kidney tissue-mimetic construct, or a lung tissue-mimetic construct.

3. The method of claim 1, wherein the tissue-mimetic construct is a skin-mimetic construct, the epithelial cells are keratinocytes and the stromal cells are fibroblasts.

4. The method of claim 1, wherein the mixed cell culture includes a scaffold.

5. The method of claim 4, wherein the scaffold is a hydrogel.

6. The method of claim 5, wherein the hydrogel comprises collagen, fibrin, agarose, hyaluronic acid, polyethylene glycol, alginate or cellulose.

7. A tissue-mimetic construct produced by the method of claim 1.

8. A method of producing an acellular scaffold containing an extracellular matrix, comprising:
a) combining epithelial cells, stromal cells and one or more carbohydrate-based macromolecules, thereby producing a mixed cell culture;
b) maintaining the cell culture under conditions in which the epithelial cells and stromal cells proliferate and produce an extracellular matrix; and
c) decellularizing the cell culture,
thereby producing an acellular scaffold containing an extracellular matrix.

9. The method of claim 8, wherein the epithelial cells are keratinocytes.

10. The method of claim 8, wherein the stromal cells are fibroblasts.

11. An acellular scaffold produced by the method of claim 8.

12. A method of producing a scaffold comprising a hydrogel that is enriched in extracellular matrix (ECM) components, the method comprising:
a) preparing an enriched extracellular matrix by culturing epithelial cells, stromal cells, or a combination thereof with one or more macromolecules having a hydrodynamic radius in the range of from about 2 nm to about 50 nm; and
b) combining the enriched extracellular matrix with a hydrogel, thereby producing a scaffold comprising a hydrogel that is enriched in ECM components.

13. The method of claim 12, wherein the hydrogel is a collagen gel, a fibrin gel, an agarose gel, a hyaluronic acid gel, a polyethylene glycol gel, an alginate gel or a cellulose gel.

14. The method of claim 12, wherein the hydrogel is an agarose gel.

15. The method of claim 12, wherein the epithelial cells are keratinocytes.

16. The method of claim 12, wherein the stromal cells are fibroblasts.

17. A scaffold produced by the method of claim 12.

18. A method of treating a condition in a subject in need thereof, comprising producing a tissue-mimetic construct according to the method of claim 1 and applying the tissue-mimetic construct to the skin of the subject, thereby treating the condition.

19. A method of assessing whether an agent is suitable for administering to a tissue, comprising:
a) contacting a tissue-mimetic construct produced according to the method claim 1 with an agent to be assessed;
b) determining whether the agent produces a desired effect on the tissue-mimetic construct compared to a control,
wherein if the agent produces a desired effect on the tissue-mimetic construct compared to a control, then the agent is suitable for administering to the tissue.

20. A method of producing a scaffold that is enriched in extracellular matrix (ECM) components, wherein the ECM components are concentrated on the surface of the scaffold, the method comprising:
a) preparing an enriched extracellular matrix by culturing epithelial cells, stromal cells, or a combination thereof with one or more macromolecules having a hydrodynamic radius in the range of from about 2 nm to about 50 nm; and
b) attaching the enriched extracellular matrix to the surface of the scaffold,
thereby producing a scaffold that is enriched in ECM components on the surface.
